(19) United States
(12) Patent Application Publication
(10) Pub. No.: US 2024/0124396 A1
(43) Pub. Date: Apr. 18, 2024

(54) AMINO LIPID COMPOUND, PREPARATION METHOD THEREFOR, AND USE THEREOF

(71) Applicant: SHENZHEN SHENXIN BIOTECHNOLOGY CO., LTD., Nanshan District, Shenzhen (CN)

(72) Inventor: Linxian Li, Shenzhen (CN)

(21) Appl. No.: 18/264,397

(22) PCT Filed: Jan. 27, 2022
(86) PCT No.: PCT/CN2022/074272

§ 371 Date: Aug. 4, 2023

(30) Foreign Application Priority Data
Feb. 7, 2021 (CN) 202110168046.4
Feb. 7, 2021 (CN) 202110178738.7

(51) Int. Cl. C07D 207/46; A61K 47/22; C07D 211/60; C07D 401/12

(52) U.S. Cl. CPC C07D 207/46; A61K 47/22; C07D 211/60; C07D 401/12

(57) ABSTRACT

The present invention relates to an amino lipid compound, namely a compound represented by chemical formula I, or a stereoisomer, tautomer, pharmaceutically acceptable salt, or prodrug thereof. Moreover, also disclosed are a preparation method for the compound and the use of the compound in delivering a genetic material and preparing a drug.

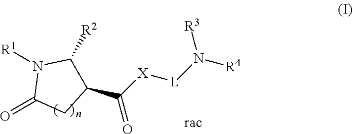

### TECHNICAL FIELD

The present invention relates to an amino lipid compound, in particular to an amino lipid compound that can be used for delivering genetic substances (also known as genes) (such as nucleic acids, e.g. mRNA) into cells, and a preparation method and use thereof.

### BACKGROUND TECHNOLOGY

Gene therapy is to deliver genes with specific genetic information to target cells by artificial means, and the expressed target proteins have the effect of regulating, treating and even curing diseases caused by congenital or acquired gene defects. Both nucleic acid and cell membrane are negatively charged. Therefore, naked nucleic acids are difficult to be directly introduced into cells, and they are easily degraded by nucleic acid-degrading enzymes in the cytoplasm, which cannot achieve the effect of gene introduction and gene therapy. Therefore, it is necessary to use external force or vector to achieve gene delivery.

The liposome nanometer particles (lipid-based nanoparticle, LNP) can encapsulate RNA vaccine, medicine and gene editing tools, realizes the delivery process in the in vivo administration engineering, and is increasingly widely applied to the delivery of gene medicines such as nucleic acid and mRNA. Stable, uniform, and particle size-controllable LNPs are prepared from cationic lipids and helper lipids (phospholipids, cholesterol, and PEGylated lipids) through micro-channel chips with defined channels, wherein the helper lipids have been commercialized, and the cationic lipids directly determine the encapsulation and delivery efficiency of mRNA, which is the core element for the LNP technology development.

Nucleic acids with different molecular weights have different requirements for the chemical structure of ionizable lipids of LNP. Once different application scenarios and delivery objects are faced, there is also a great difference between the structures of LNP vectors required for effectively delivering the nucleic acid. Therefore there is a strong industry development need to develop amino lipids with different chemical structures to effectively deliver the mRNA.

### SUMMARY OF THE INVENTION

Aiming at the shortcomings in the prior art, the present invention provides an amino lipid compound that can be used to deliver genetic substances (such as nucleic acids, e.g. mRNA) into cells, a composition comprising the same, a preparation method and use thereof.

In the first aspect, the present invention provides an amino lipid compound (i.e. a compound represented by chemical formula I), or a stereoisomer thereof, or a tautomer thereof, or a pharmaceutically acceptable salt thereof, a prodrug thereof, or a solvate thereof:

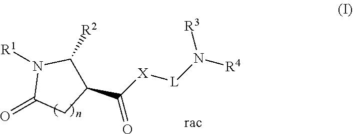

wherein:
R¹ and R² are independently to each other selected from the following structures:
a linear or branched, substituted or unsubstituted alkyl structure containing 6 to 24 carbon atoms, wherein in the substituted alkyl structure, said substituent group is a hydrocarbyl group containing 1 to 6 carbon atoms;
a linear or branched, substituted or unsubstituted alkenyl structure containing 6 to 24 carbon atoms, wherein in the substituted alkenyl structure, said substituent group is a hydrocarbyl group containing 1 to 6 carbon atoms;
a linear or branched, substituted or unsubstituted alkynyl structure containing 6 to 24 carbon atoms, wherein in the substituted alkynyl structure, said substituent group is a hydrocarbyl group containing 1 to 6 carbon atoms;
a linear or branched, saturated or unsaturated, substituted or unsubstituted acyl structure containing 4 to 24 carbon atoms, wherein in the substituted acyl structure, said substituent group is a hydrocarbyl group containing 1 to 6 carbon atoms;
R³ and R⁴ are independently to each other selected from the following structures:
a linear or branched, substituted or unsubstituted alkyl structure containing 1 to 12 carbon atoms, wherein in the substituted alkyl structure, said substituent group is a hydrocarbyl group containing 1 to 6 carbon atoms;
a linear or branched, substituted or unsubstituted alkenyl structure containing 2 to 12 carbon atoms, wherein in the substituted alkenyl structure, said substituent group is a hydrocarbyl group containing 1 to 6 carbon atoms;
a linear or branched, substituted or unsubstituted alkynyl structure containing 2 to 12 carbon atoms, wherein in the substituted alkynyl structure, said substituent group is a hydrocarbyl group containing 1 to 6 carbon atoms;
R³ and R⁴ combine with each other to form a 4 to 10-membered heterocycle, wherein the heteroatom is one or more heteroatoms of nitrogen, sulfur, and oxygen, and the heterocycle optionally comprises 1 to 6 heteroatoms;
L is selected from the following structures:
a linear or branched, substituted or unsubstituted alkylene structure containing 1 to 12 carbon atoms, said substituent group is one or more of a hydrocarbyl group, a carboxyl group, an acyl group, and an alkoxy group;
a linear or branched, substituted or unsubstituted alkenylene structure containing 2 to 12 carbon atoms, said substituent group is one or more of a hydrocarbyl group, a carboxyl group, an acyl group, and an alkoxy group;
a linear or branched, substituted or unsubstituted alkynylene structure containing 2 to 12 carbon atoms, said substituent group is one or more of a hydrocarbyl group, a carboxyl group, an acyl group, and an alkoxy group;
a 4 to 10-membered heterocycle structure, the heteroatom is one or more heteroatoms of nitrogen, sulfur, and oxygen, and the heterocycle optionally comprises 1 to 6 heteroatoms;
n=1 or 2;
X is one selected from —CH₂—, —NH—, —O—, —S—, —S(═O)—, —S(═O)₂— and —(S—S)—.

In a preferable embodiment, said R¹ is one selected from the following N6, N7, N8, N9, N10, N11, N12, N13, N14, N15, N16, N18, N19 and N20:
N6: CH₃(CH₂)₅—; N7: CH₃(CH₂)₆—; N8: CH₃(CH₂)₇—;
N9: CH₃(CH₂)₈—; N10: CH₃(CH₂)₉—; N11: CH₃(CH₂)₁₀—;
N12: CH₃(CH₂)₁₁—; N13: CH₃(CH₂)₁₂—; N14: CH₃(CH₂)₁₃—;
N15: CH₃(CH₂)₁₄—; N16: CH₃(CH₂)₁₅—; N18: CH₃(CH₂)₁₇—;

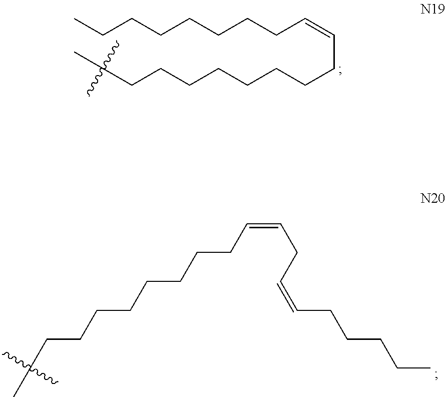 [SMILES: *CCCCCCCC/C=C\CCCCCCCC ; *CCCCCCCC/C=C\C/C=C\CCCCC]

said R² is one selected from the following A6, A7, A8, A9, A10, A11, A12, A13, A14, A15, A16, A18, A19, and A20:
A6: CH₃(CH₂)₄—; A7: CH₃(CH₂)₅—; A8: CH₃(CH₂)₆—;
A9: CH₃(CH₂)₇—; A10: CH₃(CH₂)₈—; A11: CH₃(CH₂)₉—;
A12: CH₃(CH₂)₁₀—; A13: CH₃(CH₂)₁₁—; A14: CH₃(CH₂)₁₂—;
A15: CH₃(CH₂)₁₃—; A16: CH₃(CH₂)₁₄—; A18: CH₃(CH₂)₁₆—;

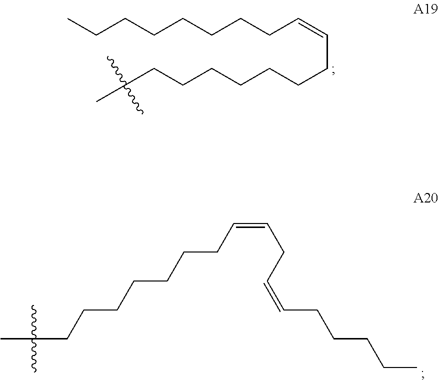 [SMILES: *CCCCCCC/C=C\CCCCCCCC ; *CCCCCCC/C=C\C/C=C\CCCCC]

—X-L-N(R³)(R⁴) is any one selected from the following O1, O2, O3, O4, O5, O6, O7, O8, O9, O10, D1, D2, D3, D4, D5, D6, D7, D8, D9, and D10:

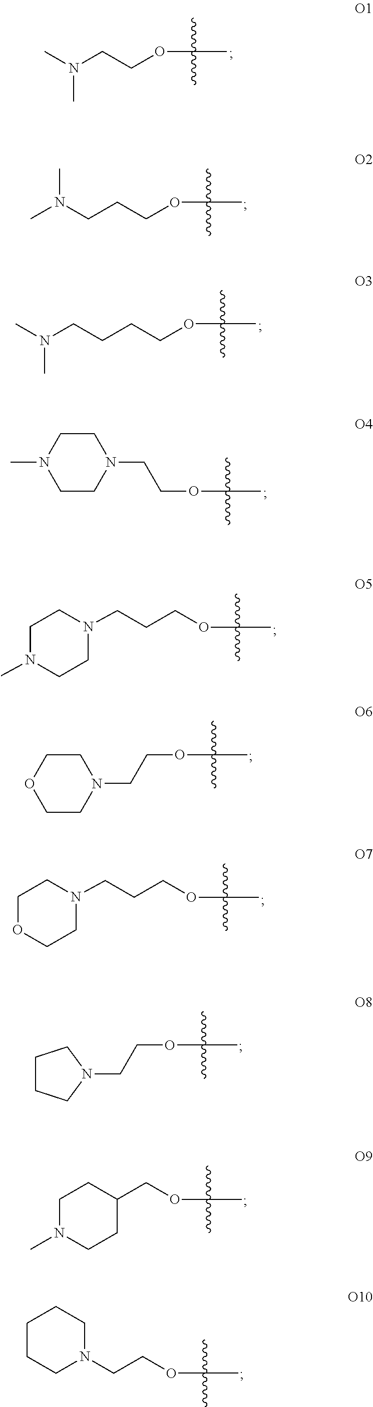

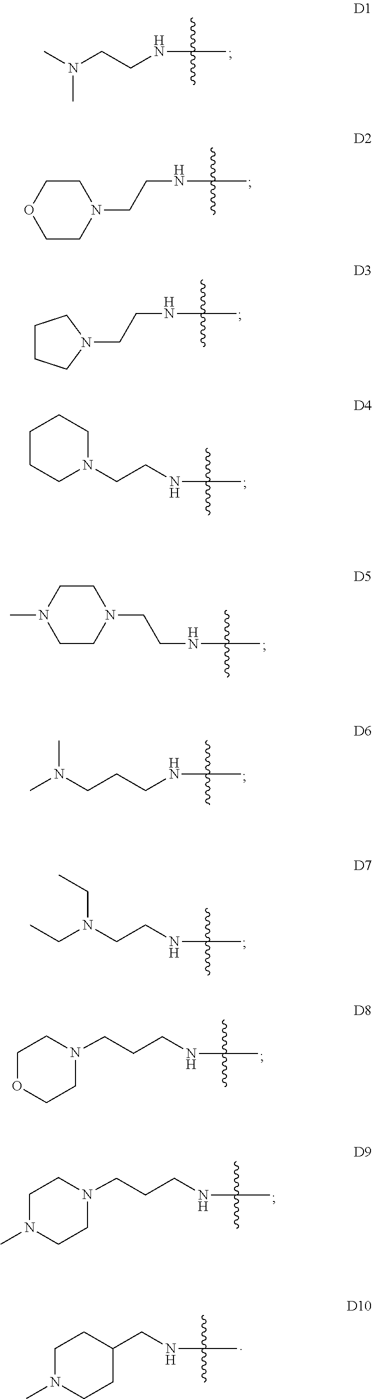

In a preferable embodiment, R¹ and R² are independently to each other selected from the following structures:
a linear or branched, substituted or unsubstituted alkyl structure containing 6 to 24 carbon atoms, wherein in the substituted alkyl structure, said substituent group is a hydrocarbyl group containing 1 to 6 carbon atoms;
a linear or branched, substituted or unsubstituted alkenyl structure containing 6 to 24 carbon atoms, wherein in the substituted alkenyl structure, said substituent group is a hydrocarbyl group containing 1 to 6 carbon atoms;
R³ and R⁴ are independently to each other selected from the following structures:
a linear or branched, substituted or unsubstituted alkyl structure containing 1 to 12 carbon atoms, wherein in the substituted alkyl structure, said substituent group is a hydrocarbyl group containing 1 to 6 carbon atoms;
R³ and R⁴ combine with each other to form a 4 to 10-membered heterocycle structure, wherein the heteroatom is one or more heteroatoms of nitrogen, sulfur, and oxygen, and the heterocycle optionally comprises 1 to 6 heteroatoms;
X is —NH— or —O—.

In a preferable embodiment, said L is a linear or branched, substituted or unsubstituted alkylene structure containing 1 to 4 carbon atoms, wherein in the substituted alkylene structure, said substituent group is a hydrocarbyl group containing 1 to 6 carbon atoms.

In a preferable embodiment, said X is —O—.

In a preferable embodiment, said X is —NH—.

In a preferable embodiment, said R¹ is one selected from the following N6, N7, N8, N9, N10, N11, N12, N13, N14, N15, N16, N18, N19 and N20:
N6: CH₃(CH₂)₅—; N7: CH₃(CH₂)₆—; N8: CH₃(CH₂)₇—;
N9: CH₃(CH₂)₈—; N10: CH₃(CH₂)₉—; N11: CH₃(CH₂)₁₀—;
N12: CH₃(CH₂)₁₁—; N13: CH₃(CH₂)₁₂—; N14: CH₃(CH₂)₁₃—;
N15: CH₃(CH₂)₁₄—; N16: CH₃(CH₂)₁₅—; N18: CH₃(CH₂)₁₇—;

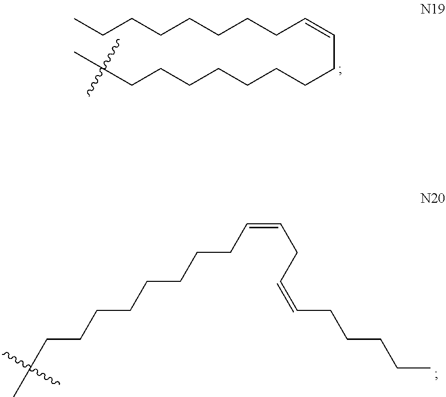 [SMILES: *CCCCCCCC/C=C\CCCCCCCC ; *CCCCCCCC/C=C\C/C=C\CCCCC]

said R² is one selected from the following A6, A7, A8, A9, A10, A11, A12, A13, A14, A15, A16, A18, A19, and A20:
A6: CH₃(CH₂)₄—; A7: CH₃(CH₂)₅—; A8: CH₃(CH₂)₆—;
A9: CH₃(CH₂)₇—; A10: CH₃(CH₂)₈—; A11: CH₃(CH₂)₉—;
A12: CH₃(CH₂)₁₀—; A13: CH₃(CH₂)₁₁—; A14: CH₃(CH₂)₁₂—;
A15: CH₃(CH₂)₁₃—; A16: CH₃(CH₂)₁₄—; A18: CH₃(CH₂)₁₆—;

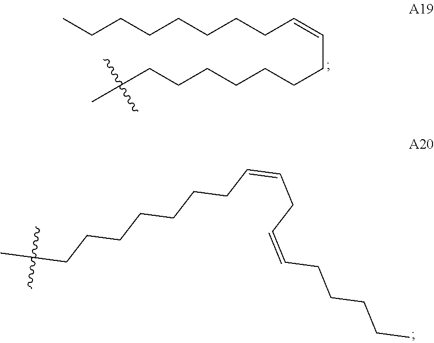 [SMILES: *CCCCCCC/C=C\CCCCCCCC ; *CCCCCCC/C=C\C/C=C\CCCCC]

and any one of N19, N20, A19 and A20 is at least contained in the molecule;
—O-L-N(R³)(R⁴) is any one selected from the following O2, O4, O5, O8, O9, and O10;

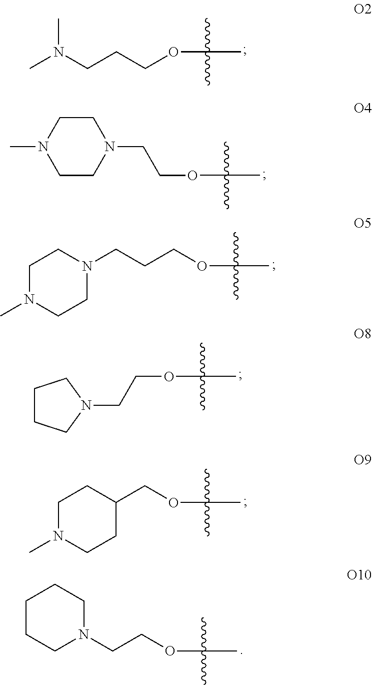

In a preferable embodiment, said R¹ is N19 or N20, said R² is any one of A10, A11, A12, A14, A15, A16, and A18.

In a preferable embodiment, said R² is A19 or A20, said R¹ is any one of N10, N11, N12, N14, N15, N16, and N18.

In a preferable embodiment, wherein said compound is any one of the following structures:

| No. | Structure |
|---|---|
| N12A12C4O2 | 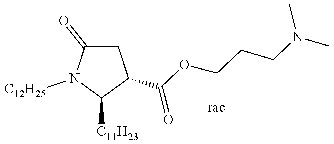 |
| N16A10C5O10 | 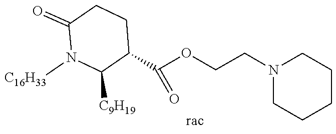 |
| 3 | 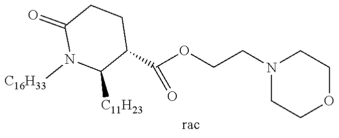 |
| 4 | 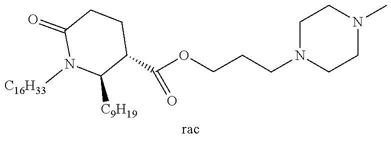 |
| 6 | 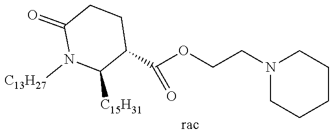 |
| 7 | 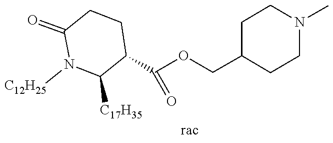 |
| N12A12C4D1 | 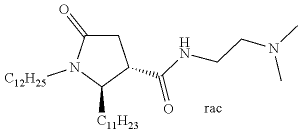 |
| 9 | 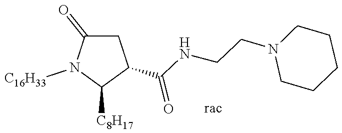 |
| 10 | 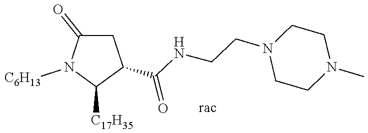 |
| 11 | 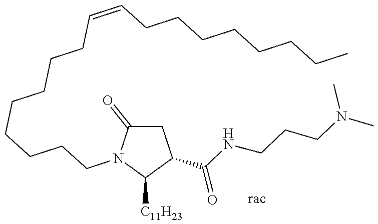 |
| 12 | 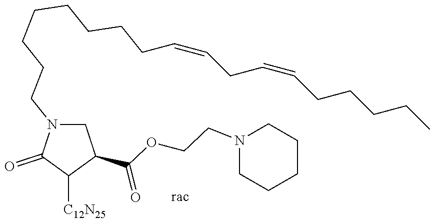 |
| 13 | 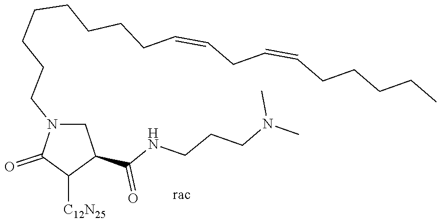 |
| 14 | 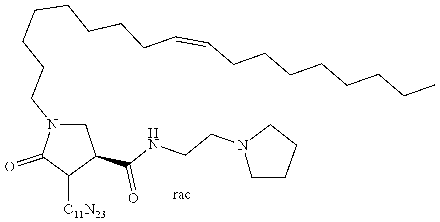 |
| 15 | 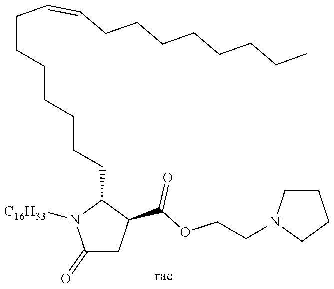 |
| 16 | 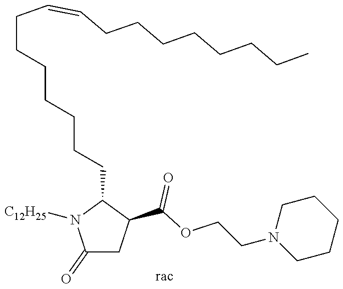 |
| N12A20C4O10 | 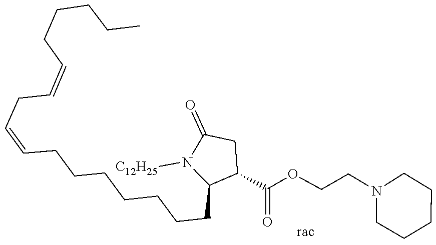 |
| 19 | 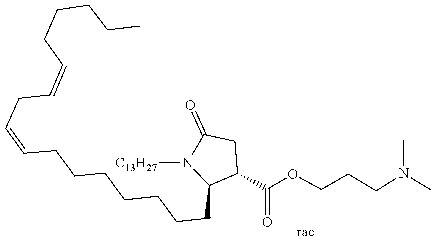 |
| 20 | 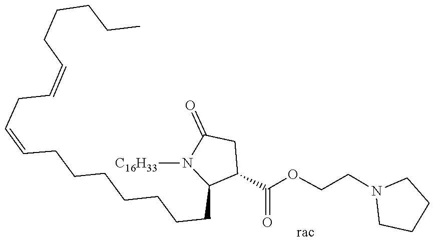 |
| Lipid 1 | 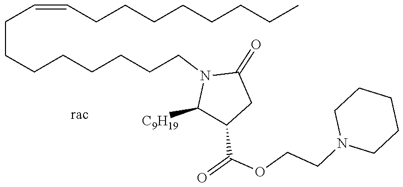 |
| Lipid 2 | 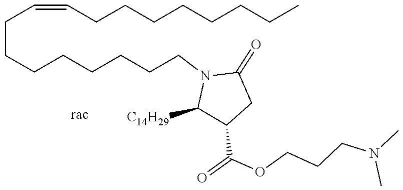 |
| Lipid 3 | 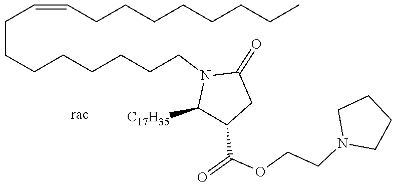 |
| Lipid 4 | 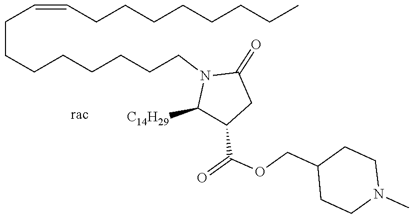 |
| Lipid 5 | 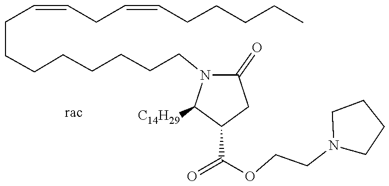 |
| Lipid 6 | 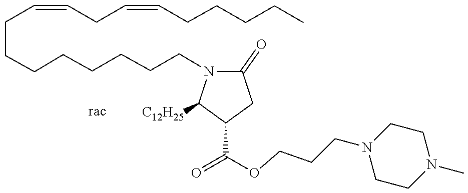 |
| Lipid 7 | 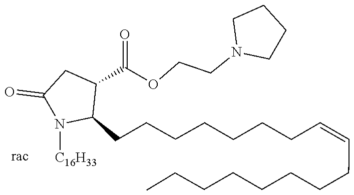 |
| Lipid 8 | 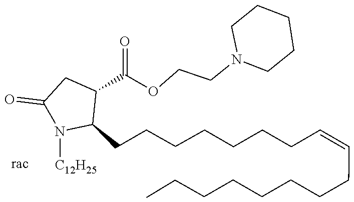 |
| Lipid 9 | 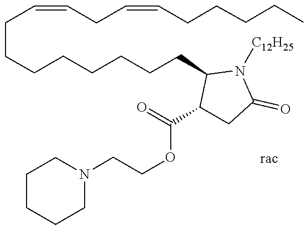 |
| Lipid 10 | 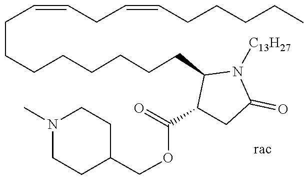 |

In the second aspect, the present invention also provides a composition that can be used for delivering genetic substances (for example nucleic acids, such as mRNA) into cells, wherein the composition comprises the aforementioned amino lipid compound (i.e. a compound represented by formula I), or a stereoisomer thereof, or a tautomer thereof, or a pharmaceutically acceptable salt thereof, a prodrug thereof, or a solvate thereof, and further comprises one or more substances of a helper lipid, a sterol, a polyethylene glycol lipid and a bioactivator.

In a preferable embodiment, the helper lipid is a non-cationic lipid, the sterol is cholesterol, and the polyethylene glycol lipid is PEG2000-DMG.

In the third aspect, the present invention also provides a process for preparing an amino lipid compound according to the present invention, which comprises the following steps:
S1, stirring compounds NH₂—R¹ and R²—CHO in a solvent to react, removing the solvent by distillation, adding a cyclic acid anhydride, warming up and reacting, and purifying to obtain a compound (II) having the following structural formula,

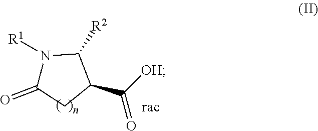

S2, reacting the compound (II) with an alcohol or amine represented by

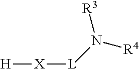

in the presence of a condensation agent to obtain a compound (I) having the following structural formula,

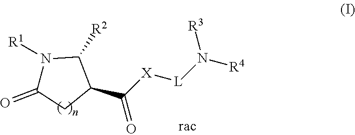

wherein each variable (such as R¹, R², R³, R⁴, n, X, and L) is defined as hereinbefore.

In the fourth aspect, the present invention also provides use of the amino lipid compound according to the present invention as the vector for delivering genetic substances in gene therapy, gene vaccine inoculation, antisense therapy, interference RNA therapy, and nucleic acid transfer.

Furthermore, the delivery vector also includes one or more substances of helper lipid, sterol, polyethylene glycol lipid, and bioactivator, which are made into lipid particles. The “lipid particles” are nano-sized substances (lipid nanoparticles) prepared by placing the amino lipid compound in an aqueous solution. Namely, a liposome is used to encapsulate a drug substance either within the lipid bilayer or in the interior aqueous space of the liposome. Liposomes are microvesicles composed of a bilayer of lipid amphipathic (amphiphilic) molecules enclosing an aqueous compartment, e.g. lipid bilayer vesicles (liposomes), multi-lamellar vesicles, micelles or the like. Liposome formation is not a spontaneous process. Lipid vesicles are formed first when lipids are placed in water and then one bilayer or a series of bilayers are formed, and each separated by water molecules. Liposomes can be created by sonicating lipid vesicles in water. The lipid bilayer is a thin membrane made of two layers of lipid molecules. The micelle is an aggregate of surfactant molecules dispersed in a liquid colloid. A typical micelle in an aqueous solution forms an aggregate with the hydrophilic head regions upon contacting with water, chelating the hydrophobic single-tail region in the micelle center.

The bioactivator is a substance that has a biological effect when introduced into a cell or host, for example, by stimulating an immune response or an inflammatory response, by exerting enzymatic activity or by complementing a mutation, and the like. The bioactivator especially is a genetic substance such as nucleic acid, peptide, protein, antibody, or small molecule, or selected from an antineoplastic agent, an antibiotic, an immunomodulator, an anti-inflammatory agent, an agent acting on the central nervous system, a polypeptide, and a polypeptoid. Said bioactivator may also be a nucleic acid, including but not limited to, messenger RNA (mRNA), antisense oligonucleotide, DNA, plasmid, ribosomal RNA (rRNA), micro RNA (miRNA), transfer RNA (tRNA), small inhibitory RNA (siRNA) and small nuclear RNA (snRNA). The bioactivator can also be an antineoplastic agent, an antibiotic, an immunomodulator, an anti-inflammatory agent, an agent acting on the central nervous system, an antigen or a fragment thereof, a protein, a peptide, a polypeptoid, a vaccine and a small-molecule or a mixture thereof. The polyethylene glycol (PEG)-lipid can help to protect the particles and their cargo from degradation in vitro and in vivo. Moreover, PEG forms a protective layer over the liposome surface and increases the circulating time in vivo. It can be used in liposome drug delivery (PEG-liposome). In this way, it can be used for transfecting multicellular tissues or organisms, which offers a novel therapeutic treatment to a patient. Said patient can be any mammal, preferably selected from the group consisting of human, mouse, rat, pig, cat, dog, horse, goat, cattle, monkey, and/or others.

Furthermore, the helper lipid is a non-cationic lipid, the sterol is cholesterol, and the polyethylene glycol lipid is PEG2000-DMG. The non-cationic lipid may contain cationic functional groups (e.g. ammonium groups) but it should contain anionic functional groups to at least neutralize the molecule. The entirety of all functional groups in the lipid molecule should be non-cationic. Liposomes consisting of a mixture of cationic amino lipids and non-cationic (neutral) phospholipids are the most effective for nucleic acid delivery into cells. The non-cationic lipid is DOPE (Dioleoylphosphatidylethanolamine) or DSPC (Distearoylphosphatidylcholine); Cholesterol is a natural component in cell membranes. It can be used to stabilize the particle and help the integration with the cell membrane. The polyethylene glycol lipid is PEG2000-DMG (1-(monomethoxypolyethylene glycol)-2,3-dimyristoyl glycerol).

In the gene therapy, the exogenous gene is introduced into the target cell through the lipid particles formed from the amino lipid compounds to correct or retrieve the disease caused by defects and abnormal genes, so as to achieve the purpose of treatment, for example, the treatment of cancer and genetic disease. The cancer is one or more of lung cancer, stomach cancer, liver cancer, esophagus cancer, colon cancer, pancreatic cancer, brain cancer, lymphatic cancer, leukaemia, and prostatic cancer, the genetic disease is one or more of hemophilia, Mediterranean anemia, and Gaucher's disease; In the vaccination, the amino lipid compound may be used to deliver an antigen or a nucleic acid encoding an antigen, and elicit an immune response against a wide variety of antigens for the treatment and/or prevention of a number of conditions such as cancer, allergy, toxicity and infection by pathogens (such as viruses, bacteria, fungi, and other pathogenic organisms). The nucleic acid in the nucleic acid transfer is any one of RNA, mRNA (messenger RNA), antisense oligonucleotide, DNA, plasmid, rRNA (ribosomal RNA), miRNA (microRNA), tRNA (transfer RNA), siRNA (small inhibitory RNA), and snRNA (small nuclear RNA), and can be used in gene therapy, gene vaccine inoculation, antisense therapy or therapy by interfering RNA in a patient. Nucleic acid has a biological effect when introduced as the bioactivator into a cell or host, for example, by stimulating an immune response or an inflammatory response, by exerting enzymatic activity, or by complementing a mutation, etc. The bioactivator includes inter alia nucleic acids, peptides, proteins, antibodies, and small molecules. Alternatively, it is a member selected from the group consisting of an antineoplastic agent, an antibiotic, an immunomodulator, an anti-inflammatory agent, an agent acting on the central nervous system, a polypeptide, or a polypeptoid. Of course, the bioactivator may also be an antineoplastic agent, an antibiotic, an immunomodulator, an anti-inflammatory agent, an agent acting on the central nervous system, an antigen or a fragment thereof, a protein, a peptide, a polypeptoid, a vaccine, and a small-molecule or a mixture thereof. Furthermore, in the use of said amino lipid compound, one or more of helper lipid, sterol, and polyethylene glycol lipid can also be added. For example, the helper lipid is a non-cationic lipid, the sterol is cholesterol, and the polyethylene glycol lipid is PEG2000-DMG (1-(monomethoxypolyethylene glycol)-2,3-dimyristoyl glycerol); the non-cationic lipid may contain a cationic functional group (e.g. an ammonium group), but it should contain an anionic functional group to at least neutralize the molecule. The entirety of all functional groups in the lipid molecule should be non-cationic. A liposome consisting of a mixture of a cationic amino lipid and a non-cationic (neutral) phospholipid is the most effective for nucleic acid delivery into cells. For example, the non-cationic lipid is DOPE (Dioleoylphosphatidylethanolamine) or DSPC (Distearoylphosphatidylcholine). Cholesterol is a natural component in cell membranes. It can be used to stabilize the particle and help the integration with the cell membrane. Polyethylene glycol lipid (PEG lipid) helps to protect the particles and their cargo from degradation in vitro and in vivo. PEG forms a protective layer over the liposome surface and increases the in vivo circulation time, and it can be used in liposome drug delivery (PEG-liposome). In this way, it can be used for transfecting multicellular tissues or organisms, which offers a novel therapeutic treatment to a patient. Said patient can be any mammal, preferably selected from the group consisting of human, mouse, rat, pig, cat, dog, horse, goat, cattle and monkey, and/or others.

The present invention also particularly provides the use of an mRNA delivery vector, wherein the delivery vector of the present invention is used as a therapeutic drug of messenger RNA and can be used in a patient in gene therapy, gene vaccine inoculation, antisense therapy, or therapy by interfering RNA. In the gene therapy, an exogenous gene is introduced into the target cell through the delivery vector of the present invention to correct or retrieve the disease caused by defects and abnormal genes, so as to achieve the purpose of treatment. It also includes the technical application of transgenesis and the like, namely, the exogenous gene is inserted into a proper receptor cell of a patient through a gene transfer technology, so that a product produced by the exogenous gene can treat certain diseases, such as common lung cancer, gastric cancer, liver cancer, esophagus cancer, colon cancer, pancreatic cancer, brain cancer, lymph cancer, blood cancer, prostate cancer and the like. Gene-edited nucleic acid substances can also be introduced for the treatment of various genetic diseases, such as hemophilia, Mediterranean anemia, Gaucher's disease, and the like. In the vaccine inoculation, the delivery vector of the present invention may be used to deliver an antigen or a nucleic acid encoding an antigen. The present invention may also be used to elicit an immune response against a wide variety of antigens for the treatment and/or prevention of a number of conditions including, but not limited to, cancer, allergy, toxicity, and infection by pathogens (such as viruses, bacteria, fungi, and other pathogenic organisms), and therefore be used to prepare a drug for nucleic acid transfer, preferably, the nucleic acid is messenger RNA (mRNA).

In a preferable embodiment, the genetic substance is any one of RNA, mRNA, antisense oligonucleotide, DNA, plasmid, rRNA, miRNA, tRNA, siRNA, and snRNA.

The present invention has the following beneficial effects: the compound of the present invention is an amino lipid compound containing a long non-polar residue; the obtained compounds all have hydrophobic character and also have hydrophilic character due to the amino group, this amphoteric character is useful for the formation of lipid particles, and meanwhile, the compounds have 5-oxopyrrolidine or 6-oxopiperidine groups, the introduction of which significantly increases the membrane fusion to enhance the mRNA release, thus promoting the synergistic improvement in the mRNA delivery, the compounds are capable of remaining stable during the in vivo circulation and being rapidly degraded in endosomes/lysosomes, and have significantly enhanced delivery efficiency. The process for preparing the amino lipid compound has the merits such as easily available raw materials, mild reaction conditions, good reaction selectivity, high reaction yield, low requirements for equipment and apparatus, and simple operation. Moreover, the amino lipid compound is used as the delivery vector of genetic substances, which significantly improves the delivery efficiency and delivery effectiveness.

### BRIEF DESCRIPTION OF THE DRAWINGS

FIG. 1 shows the humoral antibody titers produced by the delivery of OVA mRNA via the subcutaneous administration of LNPs prepared with the representative compounds N12A12C4O2, N16A10C5O10, N12A12C4D1, and N12A20C4O10 of the Examples of the present invention; and

FIG. 2 shows the humoral antibody titers produced by the delivery of OVA mRNA via the subcutaneous administration of LNPs prepared with the representative compounds Lipid 1 to Lipid 10 of the Examples of the present invention.

### DETAILED DESCRIPTION

As used herein, the following compound represented by chemical formula I exists in the form of the racemate, and the stereochemistry information shown in chemical formula I is schematic.

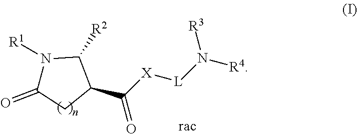

As used herein, the term “substituted” means optionally substituted, i.e., one or more hydrogen atoms attached to an atom or group are independently unsubstituted, or are substituted by one or more substituents, e.g. one, two, three or four substituents, the substituents are independently selected from: deuterium (D), halogen, —OH, mercapto, cyano, —CD₃, C₁-C₆ alkyl (preferably C₁-C₃ alkyl), C₂-C₆ alkenyl, C₂-C₆ alkynyl, cycloalkyl (preferably C₃-C₈ cycloalkyl), aryl, heterocyclyl (preferably 3-8-membered heterocyclyl), heteroaryl, arylC₁-C₆ alkyl-, heteroarylC₁-C₆ alkyl, C₁-C₆ haloalkyl, —OC₁-C₆ alkyl (preferably —OC₁-C₃ alkyl), —OC₂-C₆ alkenyl, OC₁-C₆ alkylphenyl, C₁-C₆ alkyl-OH (preferably C₁-C₄ alkyl-OH), C₁-C₆ alkyl-SH, C₁-C₆ alkyl-O—C₁-C₆ alkyl, OC₁-C₆ haloalkyl, NH₂, C₁-C₆ alkyl-NH₂ (preferably C₁-C₃ alkyl-NH₂), —N(C₁-C₆ alkyl)₂ (preferably —N(C₁-C₃ alkyl)₂), —NH(C₁-C₆ alkyl) (preferably —NH(C₁-C₃ alkyl)), —N(C₁-C₆ alkyl)(C₁-C₆ alkylphenyl), —NH(C₁-C₆ alkylphenyl), nitro, —C(O)—OH, —C(O)OC₁-C₆ alkyl (preferably —C(O)OC₁-C₃ alkyl), —CONRiRii (wherein Ri and Rii are each independently H, D or C₁-C₆ alkyl, preferably C₁-C₃ alkyl), —NHC(O)(C₁-C₆ alkyl), —NHC(O)(phenyl), —N(C₁-C₆ alkyl)C(O)(C₁-C₆ alkyl), —N(C₁-C₆ alkyl)C(O) (phenyl), —C(O)C₁-C₆ alkyl, —C(O)heteroaryl (preferably —C(O)-5-7-numbered heteroaryl), —C(O)C₁-C₆ alkylphenyl, —C(O)C₁-C₆ haloalkyl, —OC(O)C₁-C₆ alkyl (preferably —OC(O)C₁-C₃ alkyl), —S(O)₂—C₁-C₆ alkyl, —S(O)—C₁-C₆ alkyl, —S(O)₂-phenyl, —S(O)₂—C₁-C₆ haloalkyl, —S(O)₂NH₂, —S(O)₂NH(C₁-C₆ alkyl), —S(O)₂NH(phenyl), —NHS(O)₂(C₁-C₆ alkyl), —NHS(O)₂(phenyl) and —NHS(O)₂(C₁-C₆ haloalkyl), wherein each of said alkyl, cycloalkyl, phenyl, aryl, heterocyclyl and heteroaryl is optionally further substituted by one or more substituents selected from the following substituents: halogen, —OH, —NH₂, cycloalkyl, 3-8 membered heterocyclyl, C₁-C₄ alkyl, C₁-C₄ haloalkyl-, —OC₁-C₄ alkyl, —C₁-C₄ alkyl-OH, —C₁-C₄ alkyl-O—C₁-C₄ alkyl, —OC₁-C₄ haloalkyl, cyano, nitro, —C(O)—OH, —C(O)OC₁-C₆ alkyl, —CON(C₁-C₆ alkyl)₂, —CONH(C₁-C₆ alkyl), —CONH₂, —NHC(O)(C₁-C₆ alkyl), —NH(C₁-C₆ alkyl)C(O)(C₁-C₆ alkyl), —SO₂(C₁-C₆ alkyl), —SO₂(phenyl), —SO₂(C₁-C₆ haloalkyl), —SO₂NH₂, —SO₂NH(C₁-C₆ alkyl), —SO₂NH(phenyl), —NHSO₂(C₁-C₆ alkyl), —NHSO₂(phenyl) and —NHSO₂(C₁-C₆ haloalkyl). Herein when one atom or group is substituted with a plurality of substituents, the plurality of substituents may be identical or different.

As used herein, the term “hydrocarbyl” means the group remained after an aliphatic hydrocarbon loses one hydrogen atom, including straight-chain or branched-chain, saturated or unsaturated hydrocarbyl groups, including alkyl, alkenyl, and alkynyl.

As used herein, the term “acyl” refers to a hydrocarbyl-carbonyl group, preferably the acyl is C₄-C₂₄ acyl, C₆-C₁₈ acyl, C₆-C₁₂ acyl, C₆-C₁₀ acyl, C₄-C₆ acyl, C₂-C₁₂ acyl, or C₂-C₆ acyl.

As used herein, the term “alkoxy” refers to an alkyl-oxy group, preferably the alkoxy is C₁-C₁₀ alkoxy, more preferably, the alkoxy is C₁-C₆ alkoxy, most preferably, the alkoxy is C₁-C₃ alkoxy.

As used herein, the term “heterocycle” refers to a saturated or unsaturated cyclic group containing heteroatom(s) selected from N, O, S, and the like, and the heterocycle may be optionally substituted with one or more substituents.

In order to make the purposes, technical solutions, and advantages of the embodiments of the present invention clearer, the present invention will be clearly and completely described below with reference to the accompanying drawings and specific examples. Obviously, the described examples are a part of rather than all of the embodiments of the present invention. Based on the embodiments in the present invention, all other embodiments obtained by those of ordinary skill in the art without creative efforts shall fall within the protection scope of the present invention.

### Intermediate 1: 1-dodecyl-5-oxo-2-undecylpyrrolidine-3-carboxylic Acid

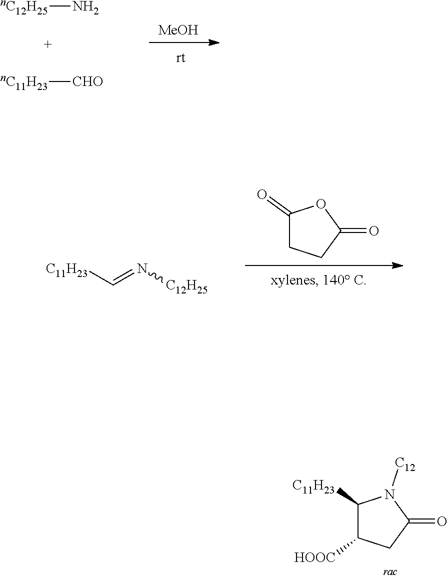

To a 250 mL reaction flask were successively added n-dodecyl amine (1.85 g, 10 mmol), n-dodecanal (1.84 g, 10 mmol), and absolute methanol (100 mL). The resulting mixture was stirred and reacted at room temperature for 12 hours. The solvent was removed by evaporation to dryness under reduced pressure. Then xylene (150 mL) and butanedioic anhydride (1.00 g, 10 mmol) were successively added. The resulting mixture was heated up to 140° C. and reacted for 10 hours. The solvent was removed by evaporation to dryness under reduced pressure, and then hexane (50 mL) was added. The resulting mixture was crystallized under stirring, filtered, washed with a small amount of hexane, and dried to produce 1-dodecyl-5-oxo-2-undecylpyrrolidine-3-carboxylic acid (3.75 g, 83%).

### Intermediate 2: 1-hexadecyl-2-nonyl-6-oxopiperidine-3-carboxylic Acid

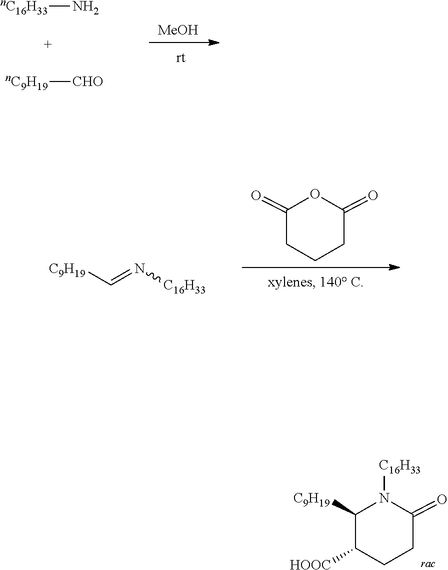

To a 250 mL reaction flask were successively added n-hexadecyl amine (2.42 g, 10 mmol), n-decyl aldehyde (1.56 g, 10 mmol), and absolute methanol (100 mL). The resulting mixture was stirred and reacted at room temperature for 12 hours. The solvent was removed by evaporation to dryness under reduced pressure. Then xylene (150 mL) and glutaric anhydride (1.14 g, 10 mmol) were successively added. The resulting mixture was heated up to 140° C. and reacted for 10 hours. The solvent was removed by evaporation to dryness under reduced pressure, and then hexane (50 mL) was added. The resulting mixture was crystallized under stirring, filtered, washed with a small amount of hexane, and dried to produce 1-hexadecyl-2-nonyl-6-oxopiperidine-3-carboxylic acid (3.51 g, 71%).

### Intermediate 3: 1-((Z)-octadeca-9-en-1-yl)-5-oxo-2-nonylpyrrolidine-3-carboxylic Acid

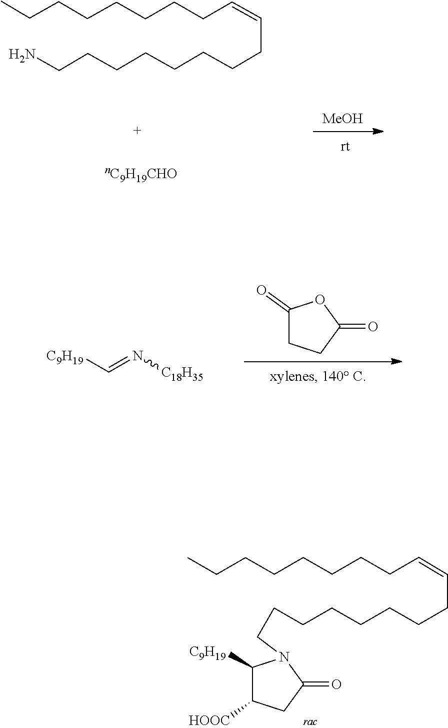

To a 250 mL reaction flask were successively added oleylamine (2.68 g, 10 mmol), n-decyl aldehyde (1.56 g, 10 mmol), and absolute methanol (100 mL). The resulting mixture was stirred and reacted at room temperature for 12 hours. The solvent was removed by evaporation to dryness under reduced pressure. Then xylene (150 mL) and butanedioic anhydride (1.00 g, 10 mmol) were successively added. The resulting mixture was heated up to 140° C. and reacted for 10 hours. The solvent was removed by evaporation to dryness under reduced pressure, and then hexane (50 mL) was added. The resulting mixture was crystallized under stirring, filtered, washed with a small amount of hexane, and dried to produce 1-((Z)-octadeca-9-en-1-yl)-5-oxo-2-nonylpyrrolidine-3-carboxylic acid (4.54 g, 90%).

### Intermediate 4: 1-dodecyl-2-((8Z,11Z)-heptadeca-8,11-dien-1-yl)-5-oxopyrrolidine-3-carboxylic Acid

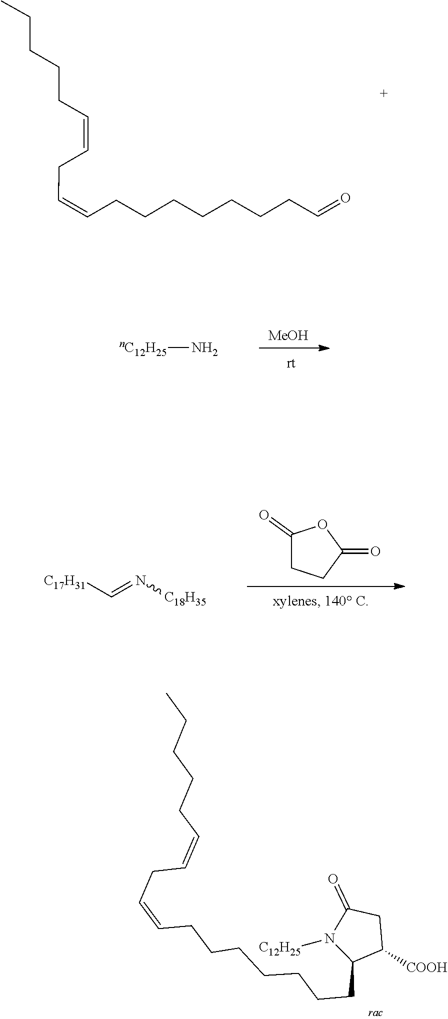

To a 250 mL reaction flask were successively added n-dodecyl amine (1.85 g, 10 mmol), cis,cis-octadeca-9,12-dienal (2.64 g, 10 mmol), and absolute methanol (100 mL). The resulting mixture was stirred and reacted at room temperature for 12 hours. The solvent was removed by evaporation to dryness under reduced pressure. Then xylene (150 mL) and butanedioic anhydride (1.00 g, 10 mmol) were successively added. The resulting mixture was heated up to 140° C. and reacted for 10 hours. The solvent was removed by evaporation to dryness under reduced pressure, and then hexane (50 mL) was added. The resulting mixture was crystallized under stirring, filtered, washed with a small amount of hexane, and dried to produce 1-dodecyl-2-((8Z,11Z)-heptadeca-8,11-dien-1-yl)-5-oxopyrrolidine-3-carboxylic acid (4.31 g, 81%).

### Intermediate 5: 1-((Z,Z)-octadeca-9,12-dien-1-yl)-5-oxo-2-undecylpyrrolidine-3-carboxylic Acid

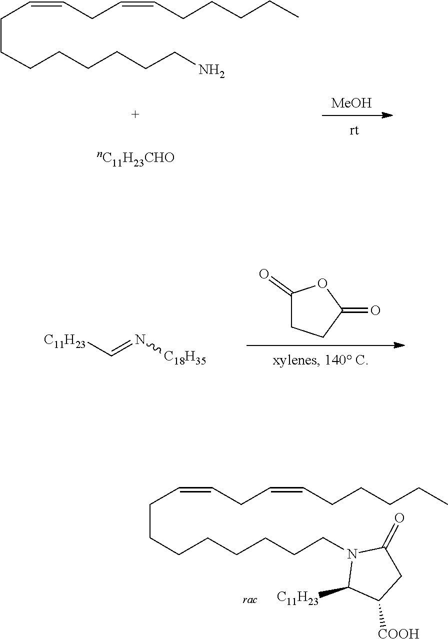

To a 250 mL reaction flask were successively added linoleylamine (2.66 g, 10 mmol), n-dodecanal (1.84 g, 10 mmol), and absolute methanol (100 mL). The resulting mixture was stirred and reacted at room temperature for 12 hours. The solvent was removed by evaporation to dryness under reduced pressure. Then xylene (150 mL) and butanedioic anhydride (1.00 g, 10 mmol) were successively added. The resulting mixture was heated up to 140° C. and reacted for 10 hours. The solvent was removed by evaporation to dryness under reduced pressure, and then hexane (50 mL) was added. The resulting mixture was crystallized under stirring, filtered, washed with a small amount of hexane, and dried to produce 1-((Z,Z)-octadeca-9,12-dien-1-yl)-5-oxo-2-undecylpyrrolidine-3-carboxylic acid (4.31 g, 81%).

### Intermediate 6: 1-dodecyl-2-((8Z)-heptadeca-8-en-1-yl)-5-oxopyrrolidine-3-carboxylic Acid

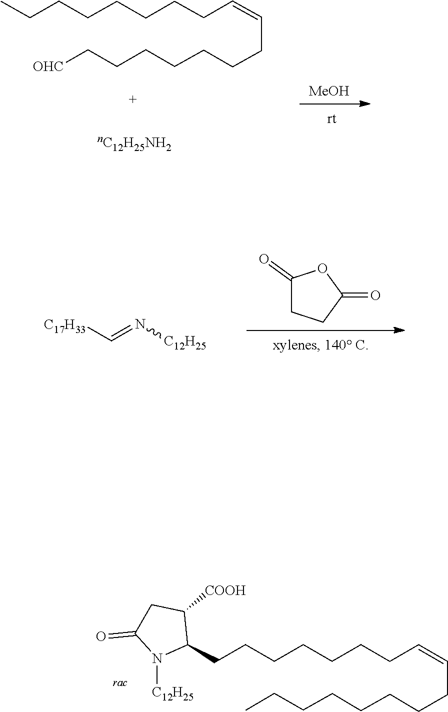

To a 250 mL reaction flask were successively added n-dodecyl amine (1.85 g, 10 mmol), cis-9-octadecenal (2.66 g, 10 mmol), and absolute methanol (100 mL). The resulting mixture was stirred and reacted at room temperature for 12 hours. The solvent was removed by evaporation to dryness under reduced pressure. Then xylene (150 mL), and butanedioic anhydride (1.00 g, 10 mmol) were successively added. The resulting mixture was heated up to 140° C. and reacted for 10 hours. The solvent was removed by evaporation to dryness under reduced pressure, and then hexane (50 mL) was added. The resulting mixture was crystallized under stirring, filtered, washed with a small amount of hexane, and dried to produce 1-dodecyl-2-((8Z)-heptadeca-8-en-1-yl)-5-oxopyrrolidine-3-carboxylic acid (4.17 g, 78%).

### Intermediate 7: 1-hexadecyl-2-((8Z,11Z)-heptadeca-8,11-dien-1-yl)-5-oxopyrrolidine-3-carboxylic Acid

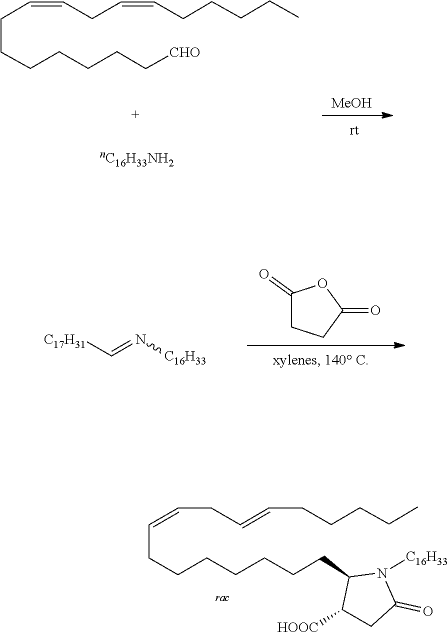

To a 250 mL reaction flask were successively added n-hexadecyl amine (2.42 g, 10 mmol), cis,cis-octadeca-9,12-dienal (2.64 g, 10 mmol), and absolute methanol (100 mL). The resulting mixture was stirred and reacted at room temperature for 12 hours. The solvent was removed by evaporation to dryness under reduced pressure. Then xylene (150 mL), and butanedioic anhydride (1.00 g, 10 mmol) were successively added. The resulting mixture was heated up to 140° C. and reacted for 10 hours. The solvent was removed by evaporation to dryness under reduced pressure, and then hexane (50 mL) was added. The resulting mixture was crystallized under stirring, filtered, washed with a small amount of hexane, and dried to produce 1-hexadecyl-2-((8Z,11Z)-heptadeca-8,11-dien-1-yl)-5-oxopyrrolidine-3-carboxylic acid (4.76 g, 81%).

### EXAMPLE 1: SYNTHESIS OF COMPOUND N12A12C4O2

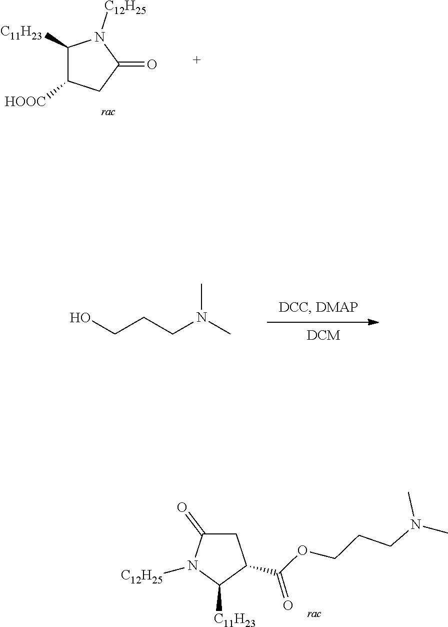

To a 250 mL reaction flask were successively added 1-dodecyl-5-oxo-2-undecylpyrrolidine-3-carboxylic acid (intermediate 1) (903 mg, 2 mmol), 3-dimethylamino-1-propanol (310 mg, 3 mmol), and dichloromethane (50 mL). The mixture was dissolved under stirring. Then dicyclohexylcarbodiimide (824 mg, 4 mmol) and 4-dimethylaminopyridine (5 mg, 0.04 mmol) were added. The resulting mixture was reacted at room temperature for 2 hours, washed with water three times, dried over anhydrous sodium sulfate, concentrated, and then purified with a flash column chromatography system (dichloromethane:methano=20:1 to 5:1) to obtain the compound N12A12C4O2 (1.03 g, 96%).

¹H NMR (400 MHz, DMSO-d6): δ4.15 (m, 2H), 3.94 (m, 1H), 3.18 (m, 2H), 2.90 (m, 1H), 2.73 (m, 1H), 2.62 (m, 1H), 2.34 (t, 2H), 2.16 (s, 6H), 1.67 (m, 2H), 1.39-1.18 (m, 40H), 0.89 (m, 6H). ESI-MS calculated for C₃₃H₆₅N₂O₃⁺ [M+H]⁺ 537.5, found 537.7

### EXAMPLE 2: SYNTHESIS OF COMPOUND N16A10C5O10

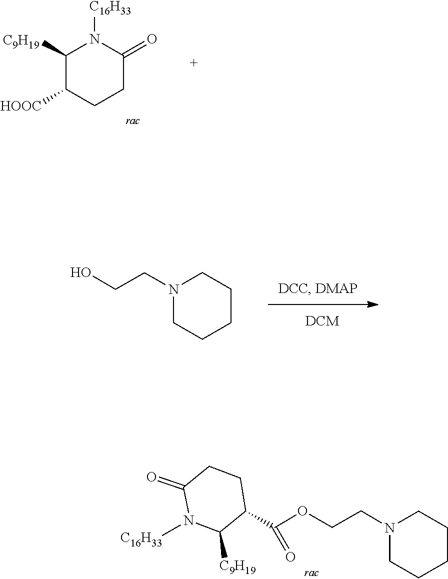

To a 250 mL reaction flask were successively added 1-hexadecyl-2-nonyl-6-oxopiperidine-3-carboxylic acid (intermediate 2) (988 mg, 2 mmol), N-ethoxypiperidine (387 mg, 3 mmol), and dichloromethane (50 mL). The mixture was dissolved under stirring. Then dicyclohexylcarbodiimide (824 mg, 4 mmol) and 4-dimethylaminopyridine (5 mg, 0.04 mmol) were added. The resulting mixture was reacted at room temperature for 2 hours, washed with water three times, dried over anhydrous sodium sulfate, concentrated, and then purified with a flash column chromatography system (dichloromethane:methanol=20:1 to 5:1) to obtain the compound N16A10C5O10 (1.03 g, 85%).

¹H NMR (400 MHz, DMSO-d6): δ4.15 (m, 2H), 3.94 (m, 1H), 3.18 (m, 2H), 2.90 (m, 1H), 2.73 (m, 1H), 2.62 (m, 1H), 2.34 (t, 2H), 2.16 (s, 6H), 1.67 (m, 2H), 1.39-1.18 (m, 40H), 0.89 (m, 6H). ESI-MS calculated for C₃₃H₆₅N₂O₃⁺ [M+H]⁺ 606.0, found 606.3

### EXAMPLE 3: SYNTHESIS OF COMPOUND N12A12C4D1

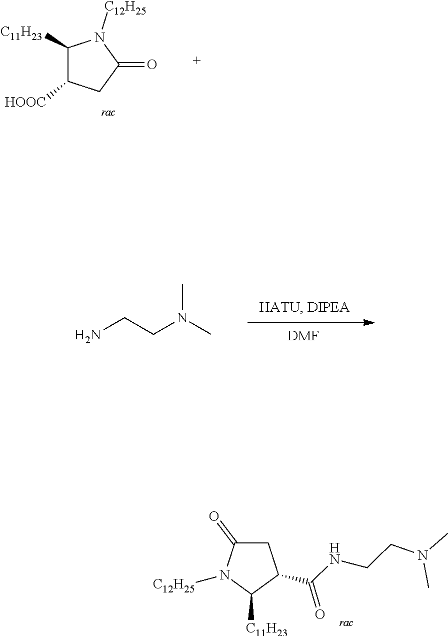

To a 250 mL reaction flask were successively added 1-dodecyl-5-oxo-2-undecylpyrrolidine-3-carboxylic acid (intermediate 1) (903 mg, 2 mmol), N,N-dimethylethylene diamine (353 mg, 4 mmol), and dichloromethane (50 mL). The mixture was dissolved under stirred. Then O-(7-azabenzotriazol-1-yl)-N,N,N′,N′-tetramethyluronium hexafluorophosphate (HATU, 1.14 g, 3 mmol) and N,N-diisopropylethylamine (516 mg, 4 mmol) were added. The resulting mixture was reacted at room temperature for 2 hours. After the completion of the reaction detected by TLC, dichloromethane (200 mL) was added. The resulting mixture was washed with water three times, dried over anhydrous sodium sulfate, concentrated, and then purified with a flash column chromatography system (dichloromethane:methanol=20:1 to 5:1) to obtain the compound N12A12C4D1 (982 mg, 94%).

¹H NMR (400 MHz, DMSO-d6): δ4.17 (m, 2H), 3.95 (m, 1H), 3.19 (m, 2H), 2.91 (m, 1H), 2.72 (m, 1H), 2.63 (m, 1H), 2.34 (t, 2H), 2.16 (s, 6H), 1.67 (m, 2H), 1.39-1.18 (m, 38H), 0.89 (m, 6H). ESI-MS calculated for C₃₂H₆₄N₃O₂⁺ [M+H]⁺ 522.5, found 522.9

### EXAMPLE 4: SYNTHESIS OF COMPOUND N12A20C4O10

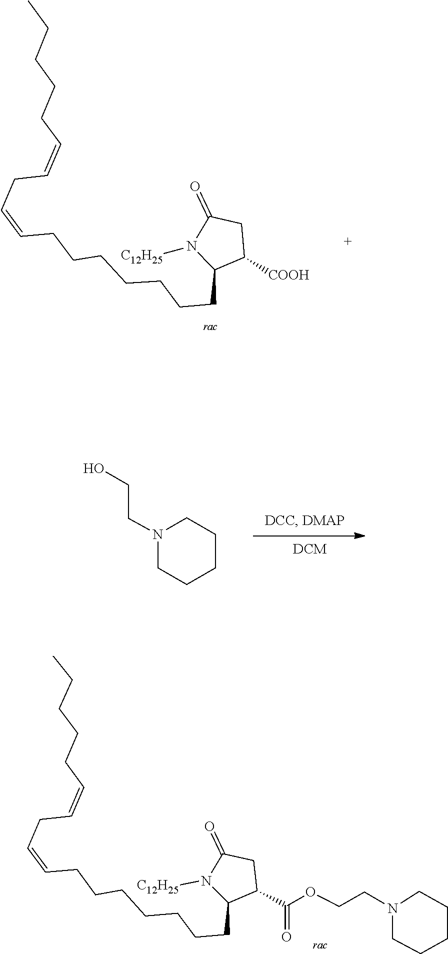

To a 250 mL reaction flask were successively added 1-dodecyl-2-((8Z,11Z)-heptadeca-8,11-dien-1-yl)-5-oxopyrrolidine-3-carboxylic acid (intermediate 4) (1064 mg, 2 mmol), N-ethoxypiperidine (387 mg, 3 mmol), and dichloromethane (50 mL). The mixture was dissolved under stirred. Then dicyclohexylcarbodiimide (824 mg, 4 mmol) and 4-dimethylaminopyridine (5 mg, 0.04 mmol) were added. The resulting mixture was reacted at room temperature for 2 hours, washed with water three times, dried over anhydrous sodium sulfate, concentrated, and then purified with a flash column chromatography system (dichloromethane:methanol=20:1 to 5:1) to obtain the compound N12A20C4O10 (1.03 g, 80%).

¹H NMR (400 MHz, DMSO-d6): δ4.15 (m, 2H), 3.94 (m, 1H), 3.18 (m, 2H), 2.90 (m, 1H), 2.73 (m, 1H), 2.62 (m, 1H), 2.34 (t, 2H), 2.16 (s, 6H), 1.67 (m, 2H), 1.39-1.18 (m, 40H), 0.89 (m, 6H). ESI-MS calculated for C₃₃H₆₅N₂O₃⁺ [M+H]⁺ 644.1, found 644.3.

### EXAMPLE 5: SYNTHESIS OF LIPID 1

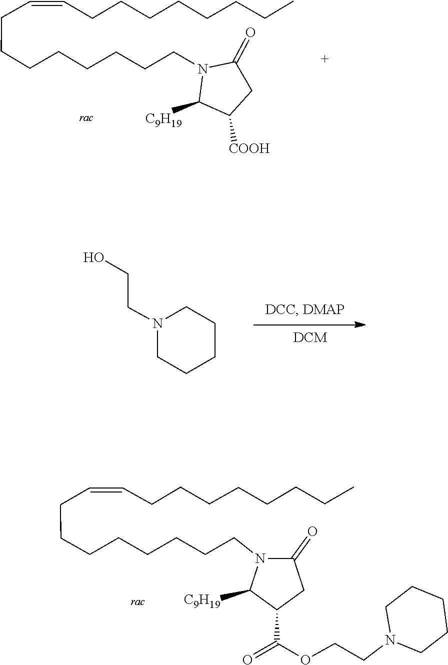

To a 250 mL reaction flask were successively added 1-((Z)-octadeca-9-en-1-yl)-5-oxo-2-nonylpyrrolidine-3-carboxylic acid (intermediate 3) (1.01 g, 2 mmol), N-ethoxypiperidine (387 mg, 3 mmol), and dichloromethane (50 mL). The mixture was dissolved under stirred. Then dicyclohexylcarbodiimide (824 mg, 4 mmol) and 4-dimethylaminopyridine (5 mg, 0.04 mmol) were added. The resulting mixture was reacted at room temperature for 2 hours, washed with water three times, dried over anhydrous sodium sulfate, concentrated, and then purified with a flash column chromatography system (dichloromethane:methanol=20:1 to 5:1) to obtain the LIPID 1 (1.16 g, 94%).

¹H NMR (400 MHz, DMSO-d6): δ5.38 (m, 2H), 4.15 (m, 2H), 3.94 (m, 1H), 3.18 (m, 2H), 2.97 (m, 2H), 2.90 (m, 1H), 2.73 (m, 1H), 2.42 (m, 4H), 2.16 (m, 4H), 1.59-1.18 (m, 46H), 0.89 (m, 6H). ESI-MS calculated for C₃₃H₆₅N₂O₃⁺ [M+H]⁺ 617.6, found 617.8.

Other amino lipid compounds were synthesized by using similar methods, and their structures were shown in Table 1. The compound named Dlin-MC3 in Table 1 was commercially available and is also known as MC3 herein.

### Assay 1

Evaluation of Luciferase mRNA In Vivo Delivery Performance of Lipid Nanoparticles Prepared from Amino Lipid Compounds
Preparation process: The delivery vector in the present invention, DSPC, cholesterol, and PEG2000-DMG were mixed and dissolved in absolute ethanol in a molar ratio of 50:10:38.5:1.5. Luciferase mRNA (Fluc mRNA) was dissolved in a sodium acetate solution (50 mM, pH=4.0). Two microinjection pumps were used, and the ratio of ethanol solution to sodium acetate solution (50 mM, pH=4.0) was controlled to be 1:3. A crude solution of lipid nanoparticles was prepared in a microflow channel chip, then dialyzed for 6 hours with 1×PBS (phosphate buffer solution) at a controlled temperature of 4° C. by using a dialysis cassette (Fisher, MWCO 20,000), and filtered with a 0.22 μm microporous filter membrane before use. The mass ratio of the amino lipid compound to luciferase mRNA (Fluc mRNA) was about 10:1.
Animal preparation: 6-week-old male BALB/c mice with body weights of about 20 g were selected, and fed in an SPF-grade feeding room. Animal experiments were strictly carried out according to the guidelines of the national health institution and the requirements of animal ethics.
In vivo delivery: 3 mice were randomly selected per group and injected with lipid nanoparticles at a dose of 0.5 mg/kg by intramuscular injection. After 6 hours, 200 μL of 10 mg/mL potassium D-fluorescein was injected into each mouse via the tail vein, and after 10 minutes, the mice were placed under an in vivo imaging system (IVIS-200, Xenogen), and the total fluorescence intensity of each mouse was observed and recorded by photographing.

Table 1 provides the expression intensities of Fluc mRNA delivered by the intramuscular injection administration of the representative amino lipid compounds with DLin-MC3 as the control (two batches of tests in total). A plurality of the amino lipid compounds had similar expression intensities to DLin-MC3, and a plurality of the amino lipid compounds had significantly better expression intensities than the positive control.

| Amino lipid | | Fluorescence | |
|---|---|---|---|
| compound | Structure | intensity | batch |
| N12A12C4O2 | 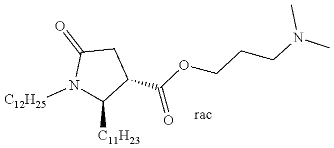 | 2.8E+06 | 1 |
| N16A10C5O10 | 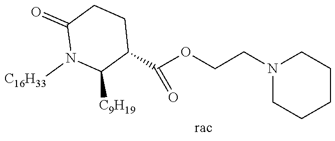 | 4.5E+06 | 1 |
| 3 | 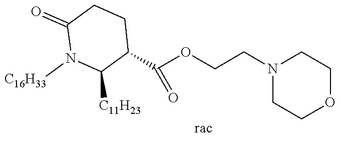 | 5.1E+06 | 1 |
| 4 | 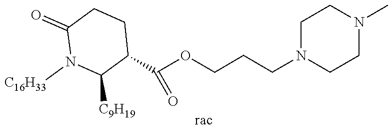 | 2.8E+06 | 1 |
| 6 | 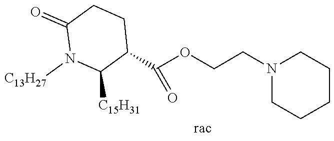 | 3.1E+05 | 1 |
| 7 | 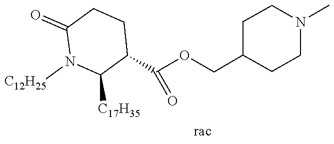 | 6.1E+05 | 1 |
| N12A12C4D1 | 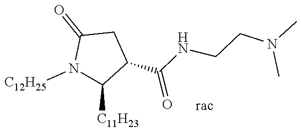 | 2.9E+07 | 1 |
| 9 | 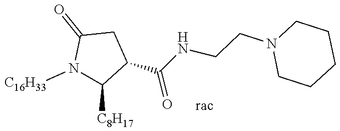 | 6.1E+06 | 1 |
| 10 | 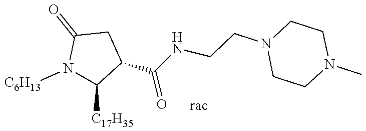 | 8.1E+05 | 1 |
| 11 | 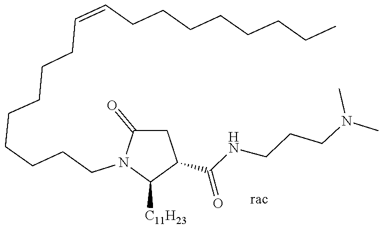 | 2.1E+06 | 1 |
| 12 | 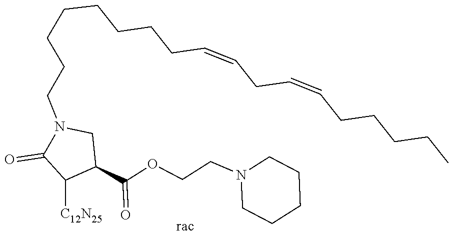 | 2.1E+06 | 1 |
| 13 | 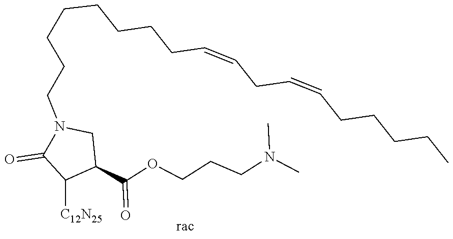 | 2.1E+06 | 1 |
| 14 | 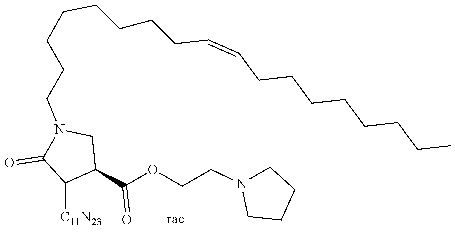 | 4.1E+06 | 1 |
| 15 | 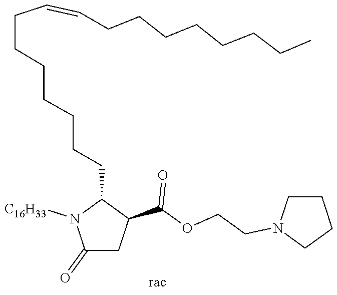 | 2.8E+07 | 1 |
| 16 | 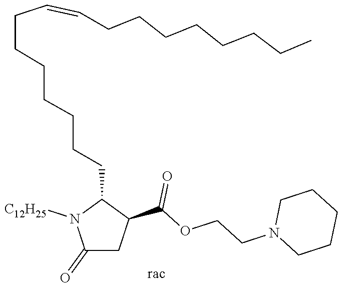 | 1.4E+07 | 1 |
| N12A20C4O10 | 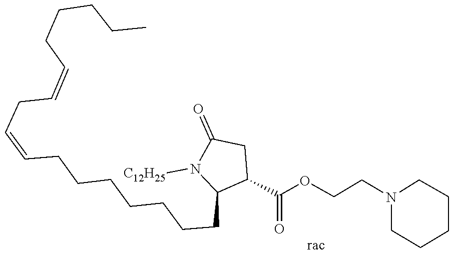 | 2.7E+06 | 1 |
| 19 | 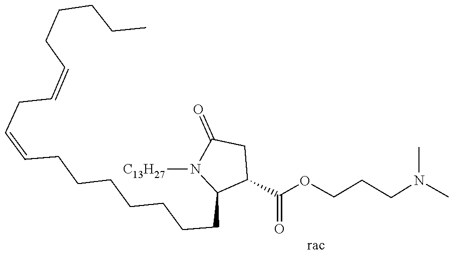 | 2.1E+06 | 1 |
| 20 | 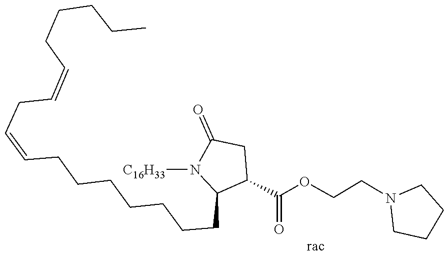 | 5.2E+06 | 1 |
| Dlin-MC3 | 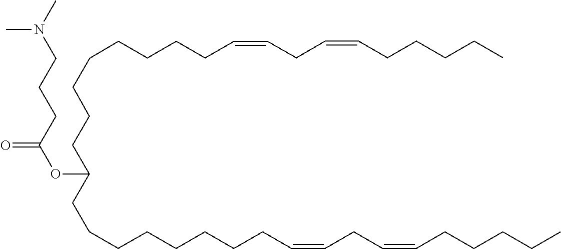 [SMILES: CCCCC/C=C\C/C=C\CCCCCCCCC(CCCCCCCC/C=C\C/C=C\CCCCC)OC(=O)CCCN(C)C] | 7.7E+06 | 1 |
| Lipid 1 | 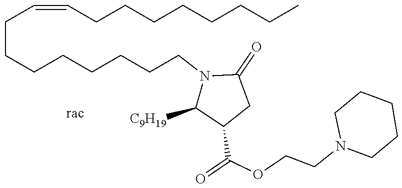 | 8.8E+06 | 2 |
| Lipid 2 | 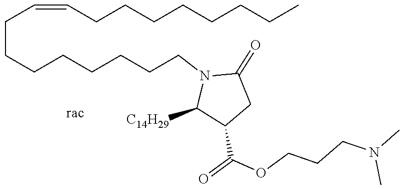 | 4.9E+06 | 2 |
| Lipid 3 | 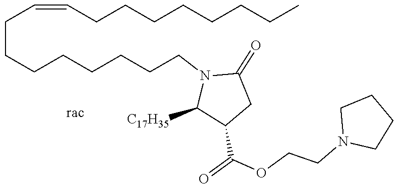 | 2.9E+07 | 2 |
| Lipid 4 | 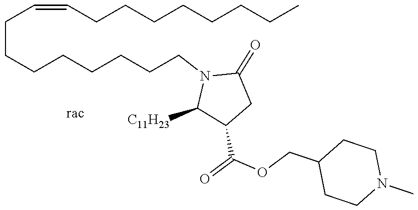 | 1.6E+07 | 2 |
| Lipid 5 | 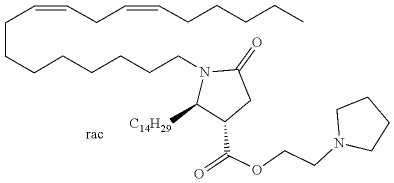 | 7.6E+06 | 2 |
| Lipid 6 | 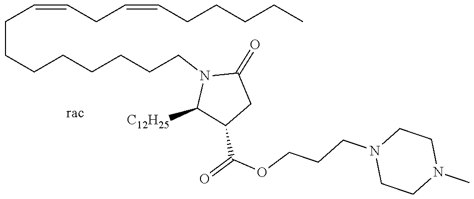 | 3.7E+06 | 2 |
| Lipid 7 | 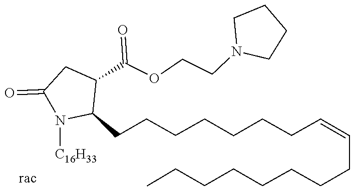 | 4.8E+07 | 2 |
| Lipid 8 | 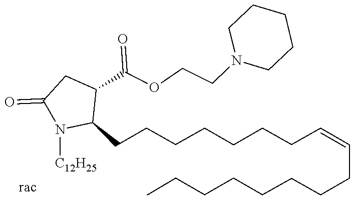 | 2.3E+07 | 2 |
| Lipid 9 | 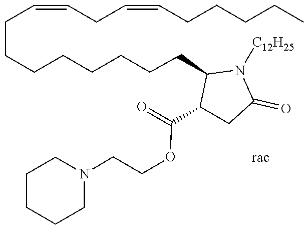 | 4.2E+06 | 2 |
| Lipid 10 | 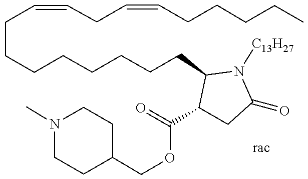 | 3.6E+06 | 2 |
| Dlin-MC3 | 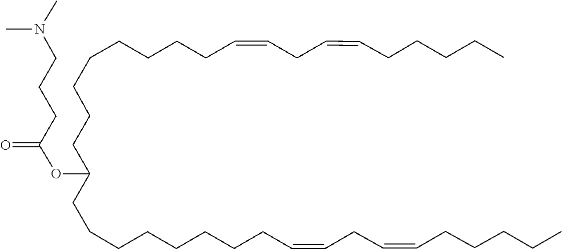 [SMILES: CCCCC/C=C\C/C=C\CCCCCCCCC(CCCCCCCC/C=C\C/C=C\CCCCC)OC(=O)CCCN(C)C] | 6.9E+06 | 2 |

### Assay 2

Evaluation of Ovalbumin mRNA In Vivo Delivery and Immunization Performance of Lipid Nanoparticles (Delivery Vector) Prepared from Amino Lipid Compounds
Preparation process: The amino lipid compound described in the present invention, DSPC, cholesterol, and PEG2000-DMG were mixed and dissolved in absolute ethanol in a molar ratio of 50:10:38.5:1.5. Ovalbumin mRNA (OVA mRNA) was dissolved in a sodium acetate solution (50 mM, pH=4.0). Two microinjection pumps were used, and the ratio of ethanol solution to sodium acetate solution (50 mM, pH=4.0) was controlled to be 1:3. A crude solution of lipid nanoparticles was prepared in a microflow channel chip, then dialyzed for 6 hours with 1×PBS at a controlled temperature of 4° C. by using a dialysis cassette (Fisher, MWCO 20,000), and filtered with a 0.22 μm microporous filter membrane before use. The mass ratio of the amino lipid compound to ovalbumin mRNA (OVA mRNA) was about 10:1.
Animal preparation: 6-week-old male BALB/c mice with body weights of about 20 g were selected, and fed in an SPF-grade feeding room. Animal experiments were strictly carried out according to the guidelines of the national health institution and the requirements of animal ethics.
In vivo delivery: 3 mice were randomly selected per group and injected with lipid nanoparticles at a dose of 0.5 mg/kg by subcutaneous injection (Day 0). After 7 days, the same amount was used for another boost (Day 7). Tail vein blood was taken on the 21st day for the serological analysis. DLin-MC3 was used as the control.
Enzyme-linked immunosorbent assay (ELISA): a flat-bottomed 96-well plate (Nunc) was pre-coated with a concentration of OVA protein of 0.5 μg protein per well in 50 mM carbonate buffer (pH 9.6), and was allowed to stay overnight at 4° C., and then blocked with 5% glycine. Anti-serums, proteins obtained from immune animals, were diluted from 10² to 10⁶ with PBS-0.05% Tween (PBS-T) at pH 7.4, added to the wells and incubated at room temperature, and placed at 37° C. for 1 hour. Horseradish peroxidase (HRP) coupled goat anti-mouse IgG was used for labeling in PBS-T-1% BSA at a dilution rate of 1:10,000. After the addition of the HRP substrate, the optical density was determined at a wavelength, and the absorbance at 450 nm was measured in an ELISA microplate reader (Bio-Rad).

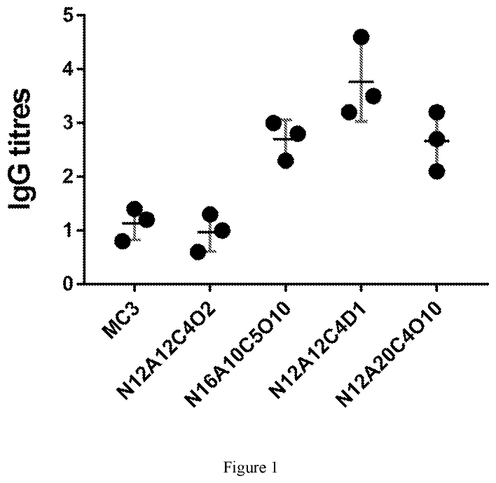

IgG antibody titers produced by the delivery of OVA mRNA via the subcutaneous administration of LNPs prepared from representative compounds N12A12C4O2, N16A10C5O10, N12A12C4D1, and N12A20C4O10 were shown in FIG. 1, wherein IgG antibody titers derived from N12A12C4O2 and DLin-MC3 were comparable, while IgG antibody titers derived from N16A10C5O10, N12A12C4D1, and N12A20C4O10 were significantly superior to that of the DLin-MC3 control group.

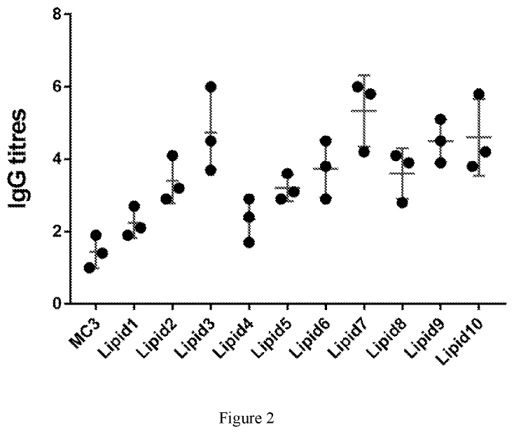

As shown in FIG. 2, IgG antibody titers produced by the delivery of OVA mRNA via the subcutaneous administration of LNPs prepared with the representative compounds Lipid 1 to Lipid 10 were significantly superior to that of the DLin-MC3 control group. It will be appreciated that modifications and variations are possible to those skilled in the art in light of the above teachings, and it is intended that the present invention cover all such modifications and variations as fall within the scope of the appended claims.

1. An amino lipid compound represented by chemical formula I, or a stereoisomer thereof, or a tautomer thereof, or a pharmaceutically acceptable salt thereof, a prodrug thereof, or a solvate thereof: 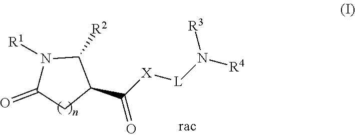
wherein:
R¹ and R² are independently to each other selected from the following structures:
a linear or branched, substituted or unsubstituted alkyl structure containing 6 to 24 carbon atoms, wherein in the substituted alkyl structure, said substituent group is a hydrocarbyl group containing 1 to 6 carbon atoms;
a linear or branched, substituted or unsubstituted alkenyl structure containing 6 to 24 carbon atoms, wherein in the substituted alkenyl structure, said substituent group is a hydrocarbyl group containing 1 to 6 carbon atoms;
a linear or branched, substituted or unsubstituted alkynyl structure containing 6 to 24 carbon atoms, wherein in the substituted alkynyl structure, said substituent group is a hydrocarbyl group containing 1 to 6 carbon atoms;
a linear or branched, saturated or unsaturated, substituted or unsubstituted acyl structure containing 4 to 24 carbon atoms, wherein in the substituted acyl structure, said substituent group is a hydrocarbyl group containing 1 to 6 carbon atoms;
R³ and R⁴ are independently to each other selected from the following structures:
a linear or branched, substituted or unsubstituted alkyl structure containing 1 to 12 carbon atoms, wherein in the substituted alkyl structure, said substituent group is a hydrocarbyl group containing 1 to 6 carbon atoms;
a linear or branched, substituted or unsubstituted alkenyl structure containing 2 to 12 carbon atoms, wherein in the substituted alkenyl structure, said substituent group is a hydrocarbyl group containing 1 to 6 carbon atoms;
a linear or branched, substituted or unsubstituted alkynyl structure containing 2 to 12 carbon atoms, wherein in the substituted alkynyl structure, said substituent group is a hydrocarbyl group containing 1 to 6 carbon atoms;
R³ and R⁴ combine with each other to form a 4 to 10-membered heterocycle structure, wherein the heteroatom is one or more heteroatoms of nitrogen, sulfur, and oxygen, and the heterocycle optionally comprises 1 to 6 heteroatoms;
L is selected from the following structures:
a linear or branched, substituted or unsubstituted alkylene structure containing 1 to 12 carbon atoms, wherein said substituent group is one or more of a hydrocarbyl group, a carboxyl group, an acyl group, and an alkoxy group;
a linear or branched, substituted or unsubstituted alkenylene structure containing 2 to 12 carbon atoms, wherein said substituent group is one or more of a hydrocarbyl group, a carboxyl group, an acyl group, and an alkoxy group;
a linear or branched, substituted or unsubstituted alkynylene structure containing 2 to 12 carbon atoms, wherein said substituent group is one or more of a hydrocarbyl group, a carboxyl group, an acyl group, and an alkoxy group;
a 4 to 10-membered heterocycle structure, the heteroatom is one or more heteroatoms of nitrogen, sulfur, and oxygen, and the heterocycle optionally comprises 1 to 6 heteroatoms;
n=1 or 2;
X is one selected from —CH₂—, —NH—, —O—, —S—, —S(═O)—, —S(═O)₂— and —(S—S)—.

2. The amino lipid compound according to claim 1, which is characterized in that said R¹ is one selected from the following N6, N7, N8, N9, N10, N11, N12, N13, N14, N15, N16, N18, N19, and N20:
N6: CH₃(CH₂)₅—; N7: CH₃(CH₂)₆—; N8: CH₃(CH₂)₇—;
N9: CH₃(CH₂)₈—; N10: CH₃(CH₂)₉—; N11: CH₃(CH₂)₁₀—;
N12: CH₃(CH₂)₁₁—; N13: CH₃(CH₂)₁₂—; N14: CH₃(CH₂)₁₃—;
N15: CH₃(CH₂)₁₄—; N16: CH₃(CH₂)₁₅—; N18: CH₃(CH₂)₁₇—; 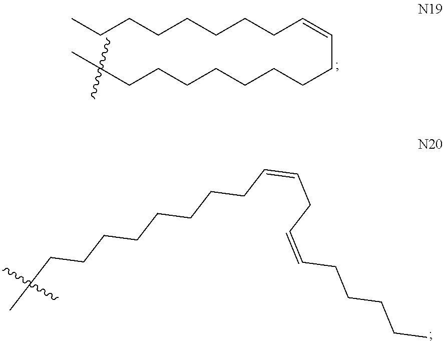 [SMILES: *CCCCCCCC/C=C\CCCCCCCC ; *CCCCCCCC/C=C\C/C=C\CCCCC]
said R² is one selected from the following A6, A7, A8, A9, A10, A11, A12, A13, A14, A15, A16, A18, A19, and A20:
A6: CH₃(CH₂)₄—; A7: CH₃(CH₂)₅—; A8: CH₃(CH₂)₆—;
A9: CH₃(CH₂)₇—; A10: CH₃(CH₂)₈—; A11: CH₃(CH₂)₉—;
A12: CH₃(CH₂)₁₀—; A13: CH₃(CH₂)₁₁—; A14: CH₃(CH₂)₁₂—;
A15: CH₃(CH₂)₁₃—; A16: CH₃(CH₂)₁₄—; A18: CH₃(CH₂)₁₆—; 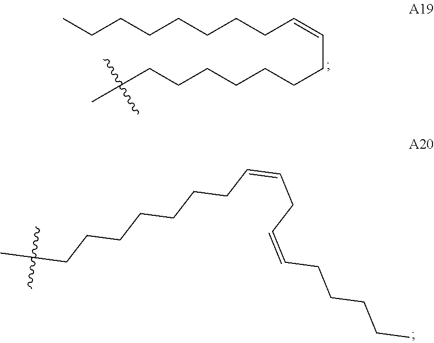 [SMILES: *CCCCCCC/C=C\CCCCCCCC ; *CCCCCCC/C=C\C/C=C\CCCCC]
—X-L-N(R³)(R⁴) is any one selected from the following O1, O2, O3, O4, O5, O6, O7, O8, O9, O10, D1, D2, D3, D4, D5, D6, D7, D8, D9, and D10:

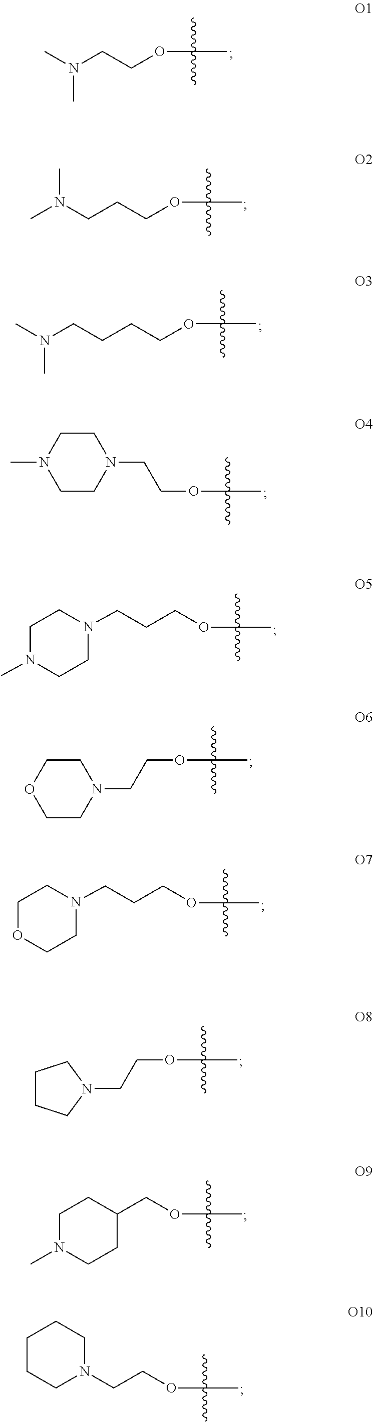

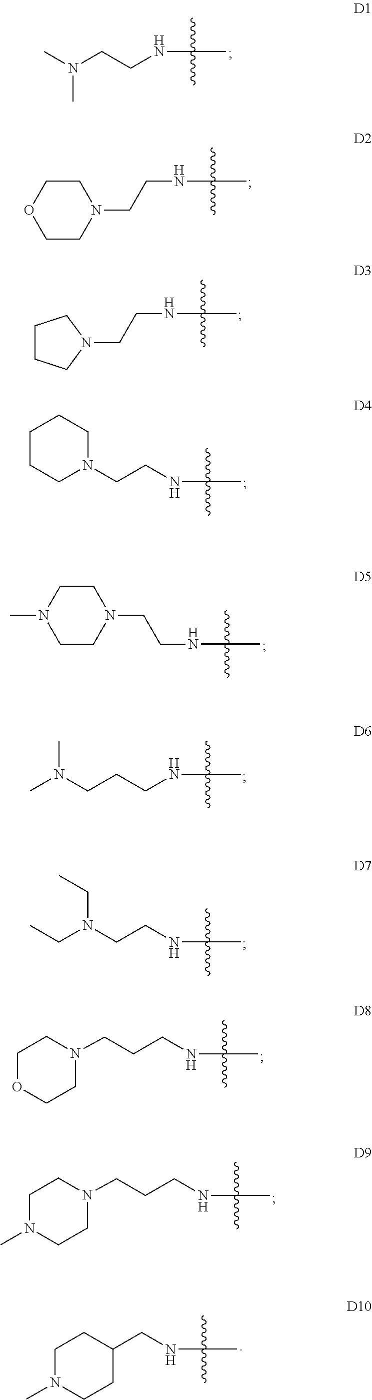

3. The amino lipid compound according to claim 1, which is characterized in that
R¹ and R² are independently to each other selected from the following structures:
a linear or branched, substituted or unsubstituted alkyl structure containing 6 to 24 carbon atoms, wherein in the substituted alkyl structure, said substituent group is a hydrocarbyl group containing 1 to 6 carbon atoms;
a linear or branched, substituted or unsubstituted alkenyl structure containing 6 to 24 carbon atoms, wherein in the substituted alkenyl structure, said substituent group is a hydrocarbyl group containing 1 to 6 carbon atoms;
R³ and R⁴ are independently to each other selected from the following structures:
a linear or branched, substituted or unsubstituted alkyl structure containing 1 to 12 carbon atoms, wherein in the substituted alkyl structure, said substituent group is a hydrocarbyl group containing 1 to 6 carbon atoms;
R³ and R⁴ combine with each other to form a 4 to 10-membered heterocycle structure, wherein the heteroatom is one or more heteroatoms of nitrogen, sulfur, and oxygen, and the heterocycle optionally comprises 1 to 6 heteroatoms;
X is —NH— or —O—.

4. The amino lipid compound according to claim 1, which is characterized in that said L is a linear or branched, substituted or unsubstituted alkylene structure containing 1 to 4 carbon atoms, wherein in the substituted alkylene structure, said substituent group is a hydrocarbyl group containing 1 to 6 carbon atoms.

5. The amino lipid compound according to claim 1, wherein said X is —O—.

6. The amino lipid compound according to claim 1, wherein
said R¹ is one selected from the following N6, N7, N8, N9, N10, N11, N12, N13, N14, N15, N16, N18, N19, and N20:
N6: CH₃(CH₂)₅—; N7: CH₃(CH₂)₆—; N8: CH₃(CH₂)₇—;
N9: CH₃(CH₂)₈—; N10: CH₃(CH₂)₉—; N11: CH₃(CH₂)₁₉—;
N12: CH₃(CH₂)₁₁—; N13: CH₃(CH₂)₁₂—; N14: CH₃(CH₂)₁₃—;
N15: CH₃(CH₂)₁₄—; N16: CH₃(CH₂)₁₅—; N18: CH₃(CH₂)₁₇—; 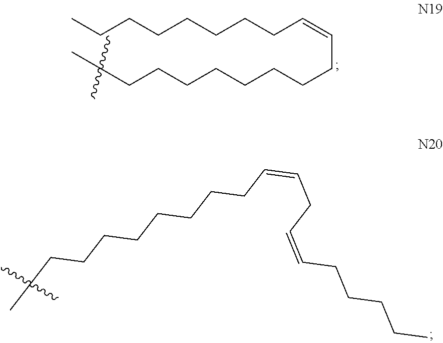 [SMILES: *CCCCCCCC/C=C\CCCCCCCC ; *CCCCCCCC/C=C\C/C=C\CCCCC]
said R² is one selected from the following A6, A7, A8, A9, A10, A11, A12, A13, A14, A15, A16, A18, A19, and A20:
A6: CH₃(CH₂)₄—; A7: CH₃(CH₂)₅—; A8: CH₃(CH₂)₆—;
A9: CH₃(CH₂)₇—; A10: CH₃(CH₂)₈—; A11: CH₃(CH₂)₉—;
A12: CH₃(CH₂)₁₀—; A13: CH₃(CH₂)₁₁—; A14: CH₃(CH₂)₁₂—;
A15: CH₃(CH₂)₁₃—; A16: CH₃(CH₂)₁₄—; A18: CH₃(CH₂)₁₆—; 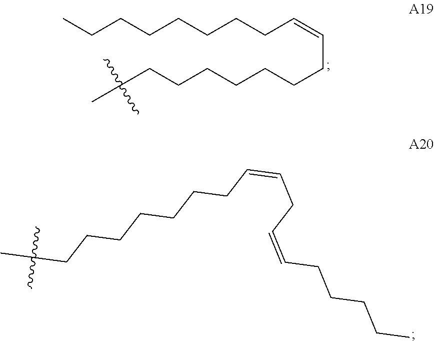 [SMILES: *CCCCCCC/C=C\CCCCCCCC ; *CCCCCCC/C=C\C/C=C\CCCCC]
and any one of N19, N20, A19 and A20 is at least contained in the molecule;
—O-L-N(R³)(R⁴) is any one selected from the following O2, O4, O5, O8, O9, and O10;

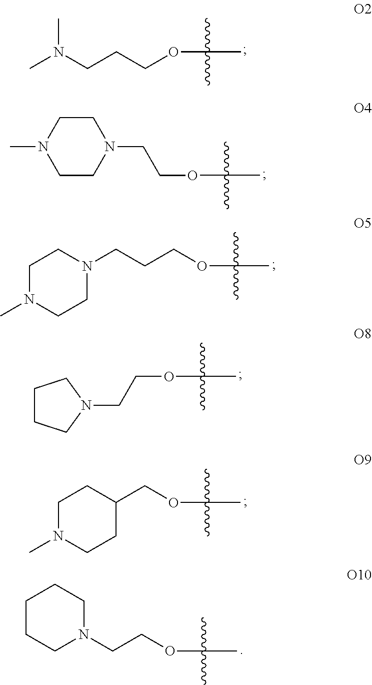

7. The amino lipid compound according to claim 1, wherein
when said R¹ is N19 or N20, said R² is any one of A10, A11, A12, A14, A15, A16, and A18; or
when said R² is A19 or A20, said R¹ is any one of N10, N11, N12, N14, N15, N16, and N18.

8. The amino lipid compound according to claim 1, wherein said amino lipid compound is:

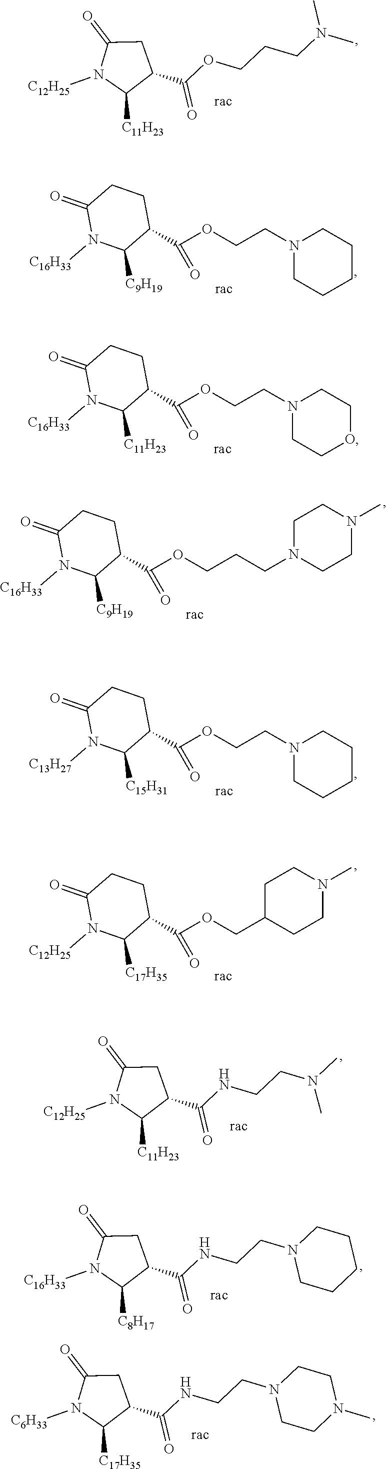

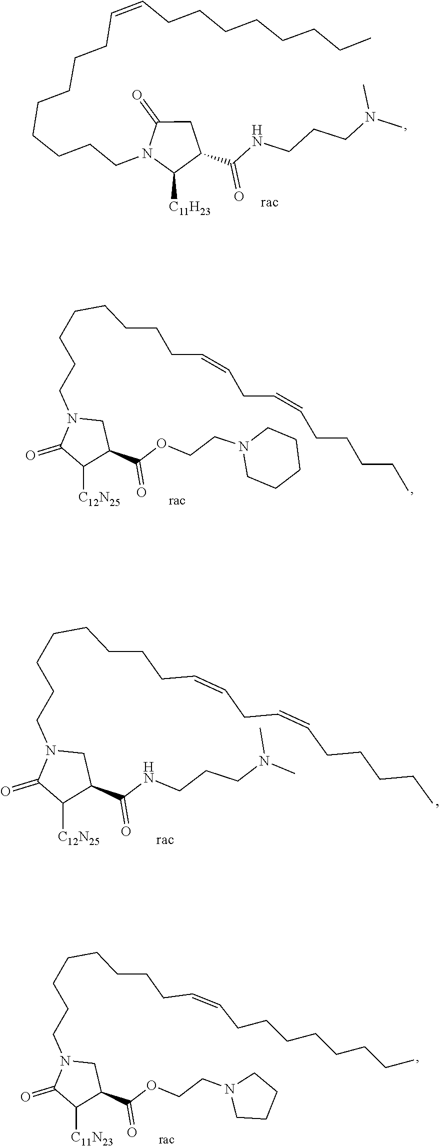

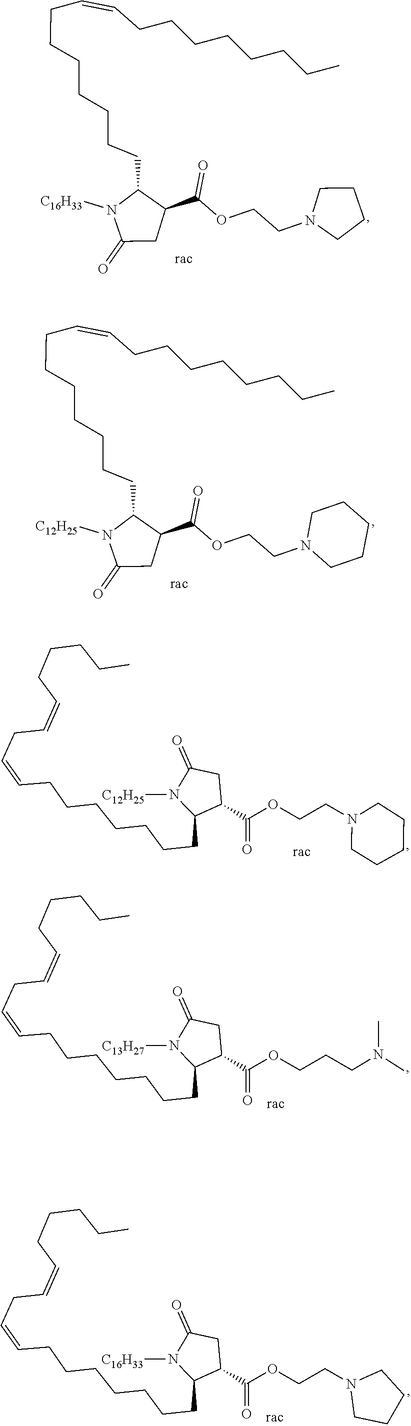

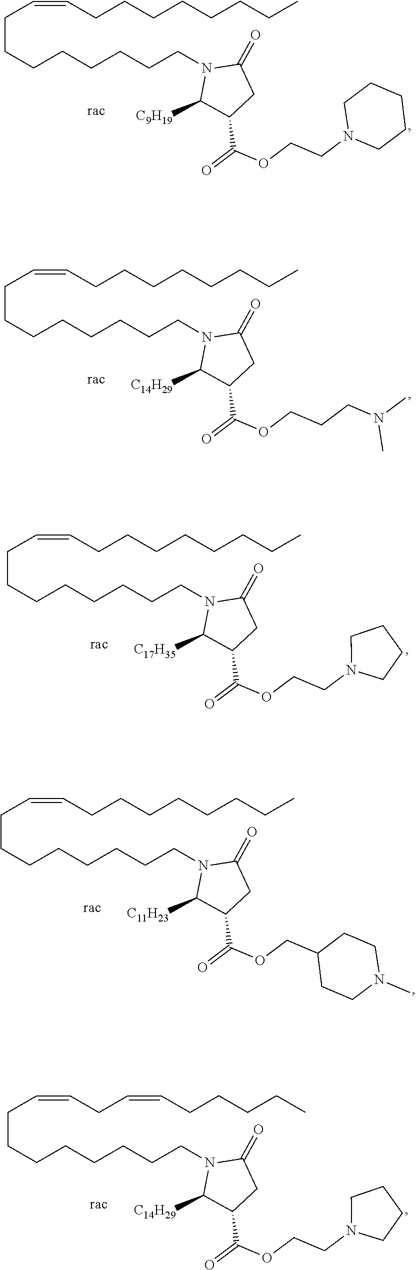

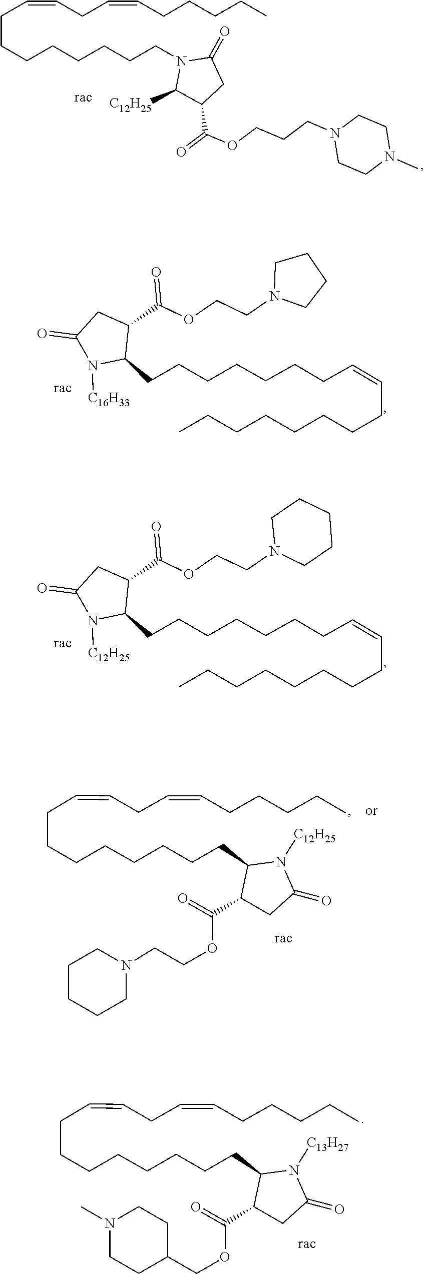

9. A process for preparing the amino lipid compound according to claim 1, which comprises the following steps:
S1, stirring compounds NH₂—R¹ and R²—CHO in a solvent to react, removing the solvent by distillation, adding a cyclic acid anhydride, warming up and reacting, and purifying to obtain a compound (II) having the following structural formula, 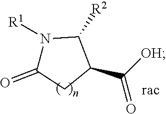
S2, reacting compound (II) with an alcohol or amine represented by 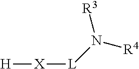
in the presence of a condensation agent to obtain a compound (I) having the following structural formula, 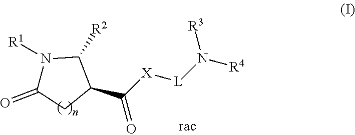
wherein:
wherein each variable such as R¹, R², R³, R⁴, n, X, and L is defined as in claim 1.

10. A composition for delivering genetic substances (for example nucleic acids, such as mRNA) into cells, wherein the composition comprises the amino lipid compound according to claim 1, preferably said composition further comprises one or more substances of a helper lipid (e.g. non-cationic lipid), a sterol (e.g. cholesterol), a polyethylene glycol lipid (e.g. PEG2000-DMG) and a bioactivator.

11. The composition according to claim 10, wherein the composition is present in the form of lipid particles.
